(19) United States
(12) Patent Application Publication
(10) Pub. No.: US 2014/0087397 A1
(43) Pub. Date: Mar. 27, 2014

(54) BIOMARKERS FOR FANCONI ANEMIA

(71) Applicants: Lindsey Romick-Rosendale, Oxford, OH (US); Susanne Wells, Cincinnati, OH (US)

(72) Inventors: Lindsey Romick-Rosendale, Oxford, OH (US); Susanne Wells, Cincinnati, OH (US)

(73) Assignee: Children's Hospital Medical Center, Cincinnati, OH (US)

(21) Appl. No.: 14/034,197

(22) Filed: Sep. 23, 2013

Related U.S. Application Data
(60) Provisional application No. 61/705,396, filed on Sep. 25, 2012.

(51) Int. Cl. G01N 33/50

(52) U.S. Cl. CPC G01N 33/5091
USPC 435/7.92; 436/173; 435/29

(57) ABSTRACT

A method for adjusting therapy given to a patient having cancer by diagnosing Fanconi Anemia (FA), and/or predicting bone marrow failure (BMF) or predicting cancer susceptibility in the patient by determining a level of at least one biomarker. In one embodiment the biomarker is at least one of 3-hydroxyisovalerate, acetate, isopropanol, glycerol-3-phosphocholine, choline, histamine, glutamate, alanine, glycine, isoleucine, lysine, phenylalanine, threonine, tyrosine, vitamin A, vitamin D, cholesterol, ketone bodies, acetone, and/or acetoacetate/acetoacetaldehyde. The biomarker level is assessed in a biological sample obtained from the patient, and an altered level of the biomarker, increased or decreased, compared to a family member not having FA or to a healthy control subject, is indicative of FA and/or predictive of BMF and cancer susceptibility. The information is used to adjust the patient's therapy based on the diagnosis of FA and/or prediction of BMF and cancer susceptibility in the patient.

This application claims priority from U.S. application Ser. No. 61/705,396, filed on Sep. 25, 2012, which is incorporated by reference in its entirety.

This invention was made with government support under Grant CA102357 awarded by the Public Health Service. The government has certain rights in the invention.

A method for adjusting therapy provided to a patient either diagnosed with or at risk for a DNA-repair defect or with cancer, such as a risk for cancer based on a diagnosis of Fanconi Anemia (FA). The method involves determining, in a biological sample of the patient, the level of at least one biomarkers or a panel of biomarkers that is/are altered compared to the level of the same biomarker(s) from a biological sample of a control individual such that the alteration is indicative of FA.

Early identification of biomarkers for cancer cells such as squamous cell carcinoma (SCC), particularly head and neck squamous cell carcinoma (HNSCC) subsequently analyzed, that identify defects in the FA pathway which under normal conditions result in DNA repair, permit altered or alternative patient treatments resulting in more favorable patient outcomes.

Traditional cancer therapies such as chemotherapy and radiation result in high morbidity and mortality in FA patients, due to their global hypersensitivity to DNA damage. It is presumed that FA tumors are exquisitely sensitive to conventional therapies such as chemotherapy and radiation, but the FA patient's hypersensitivity to DNA damage significantly limits their effectiveness, with cisplatin therapy being one example. The only curative therapy for hematologic FA abnormalities, a bone marrow transplant (BMT), causes significant morbidity and mortality in FA patients from the genotoxic conditioning regimens and the transplant itself.

The disclosed method thus involves detection of a biomarker or a biomarker panel or signature early in a patient's treatment that permits alternative and more tailored patient therapies, yielding a more favorable patient outcome. The method diagnoses FA using the disclosed biomarkers and/or biomarker signatures, and subsequently adjusts or modifies solid tumor treatment. In embodiments, modifying or adjusting treatment includes dosage, intensity, such as with radiation therapy, length of treatment, frequency of treatment, and inclusion or elimination of any additional treatments, e.g., decreasing treatment duration or intensity because of FA cells' hypersensitivity.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates the Fanconi Anemia (FA) pathway.

FIG. 2 is a flow chart outlining steps involved in one NMR-based metabonomics method.

FIGS. 3A, B, C, and D show Western blots (FIG. 3A), invasion (FIG. 3B), cell cycle profiles (FIG. 3C), and score plots and heat map profiles (FIG. 3D) from knockdown of FANCD2 (FANCD2sh) in a sporadic HNC cell line compared to non-targeting shRNA (NTsh) control.

FIGS. 4A, B, C, D, E, F, and G show metabolic profiling analysis in various cell lines in the presence or absence of FANCA expression.

FIG. 5 shows metabolic profiling results of FANCD2 knockout mouse tongues compared to FANCD2 wild type mice.

FIGS. 6A and 6B show a complete (FIG. 6A) and portion (FIG. 6B) nuclear magnetic resonance (NMR) spectrum of human urine samples.

FIG. 7 shows attenuated lipid metabolism gene expression in FANCD2sh squamous cell carcinoma 1 (SCC1) cells.

FIG. 8 shows levels of cholesterol and glycero-3-phosphocholine levels in FANCD2sh immortalized keratinocytes.

Patients with FA are at increased risk for cancer in general, and for certain types of cancers in particular. It is known that FA patient have accelerated transformation of hematopoietic cells resulting in leukemia, and have accelerated transformation of keratinocytes resulting in SCC. In one embodiment, the inventive method assess levels of biomarkers in patients at risk for or diagnosed with FA, compared to a control, and adjusts or alters the patient's existing cancer therapy based on the results. In one embodiment, the inventive method assess levels of biomarkers in patients at risk for or diagnosed with other DNA repair deficiencies, compared to a control, and adjusts or alters the patient's existing cancer therapy based on the results. In one embodiment, the method assesses an elevated risk of cancer susceptibility in a healthy individual, compared to the general population, and instructus preventative measures based on the results, e.g., frequent cancer screens. The method determines, in a sample of a patient's biological fluid, at least one biomarker that, individually or collectively, is indicative of FA. Such early identification of biomarkers for cancer cells, such as head and neck squamous cell carcinoma, subsequently analyzed, that identify FA pathway defects, can result in alternative chemotherapeutic and/or irradiation methods of treatment. Also as subsequently analyzed, because FA deficient cells are hypersensitive to DNA damage, low dose clastogenic treatments may be effective for sporadic SCC with FA mutations. Identification and diagnosis of FA in cancer patients is particularly useful where achieving tumor eradication, while minimizing toxicity, is a difficult balance.

A biomarker may be biochemical entities including precursors, breakdown proteins, derivatives, modified (e.g., glycosylated, phosphorylated, etc.) forms, metabolites, etc. of proteins including amino acids and peptides, lipids, vitamins, etc. The patent assayed may be in a normal unstressed condition, or may be in a stressed condition, e.g., having undergone a fast for at least twelve or eighteen hours, etc. Biomarkers include 3-hydroxyisovalerate, acetate, isopropanol, glycerol-3-phosphocholine, choline, histamine, glutamate, alanine, glycine, isoleucine, lysine, phenylalanine, threonine, tyrosine, fat-soluble vitamins A and D, cholesterol, ketone bodies, acetone, and/or acetoacetate/acetoacetaldehyde. Any biological sample may be assayed, e.g., cells, tissues, blood including plasma and/or serum, urine, feces, cerebrospinal fluid, saliva, lavage fluid, intrathecal fluid, etc. The term biological sample includes any or all of the above unless a specific source, e.g., urine, is stated. An altered level of the biomarker(s), compared to a control patient not having either cancer or FA i.e., a healthy control patient is indicative or suggestive of FA. In one embodiment, the confidence level of the diagnosis of FA is increased when the number of biomarkers having altered levels is increased.

This information is applied to increase, decrease, alter, combine, etc. dose, type, duration, extent, etc. of the patient's present cancer therapy based on the diagnosis of FA. The cancer therapy may be targeted or general, and may include surgical therapy, radiation therapy, chemotherapy, immunotherapy, etc. As subsequently analyzed and explained, one type of patient which the invention has particular application is a patient that has been diagnosed with or is at risk for squamous cell carcinoma (SCC). As subsequently analyzed and explained, one type of patient for which the invention has particular application is a patient that has or is at risk for bone marrow failure (BMF).

The group of diagnostic or suggestive biomarkers for FA may be termed an FA biomarker panel or FA biomarker signature. One panel includes biomarkers that participate in biochemical reactions or process involved in the metabolism of lipids. This may be further parsed as biomarkers involved in the metabolism of specific lipids, such as cholesterol. The patient may be required to endure a fast sufficient to generate a state of metabolic ketosis. One exemplary but not limiting biomarker panel includes at least two of 3-hydroxyisovalerate, acetate, isopropanol, glycerol-3-phosphocholine, phosphocholine, choline, and/or histamine. One exemplary but not limiting biomarker panel includes at least two of glutamate, alanine, glycine, isoleucine, lysine, phenylalanine, threonine, and/or tyrosine.

Biomarkers may be determined by any method. One method is nuclear magnetic resonance (NMR). A biological fluid sample from a control patient is assayed by NMR for a least one biomarker, and that level is compared to the level of the same biomarker(s) from a biological fluid in a FA patient. The biological samples assayed from the control and FA patient should be the same, e.g., control individual urine compared to FA patent urine, or control individual plasma compared to FA patient plasma. The results obtained by comparing the peak size of each biomarker from the control individual, versus the peak size of each biomarker from the FA patient, are than assessed. Treatment of the FA patient is altered, or not, as previously described, based on the difference. Depending on the degree of variation of the particular biomarker within the general population, even a small difference in a stable biomarker may be useful. The assessment can be incorporated into a kit using biochemical assays known in the art to determine the concentration of the biomarker(s). For example, known biochemical assays exist for a signature panel of, e.g., 3-hydroxyisovalerate, acetate, isopropanol, glycerol-3-phosphocholine, phosphocholine, choline, and histamine. The assays may be adapted or adaptable, as known in the art, depending upon the biological fluid, etc.

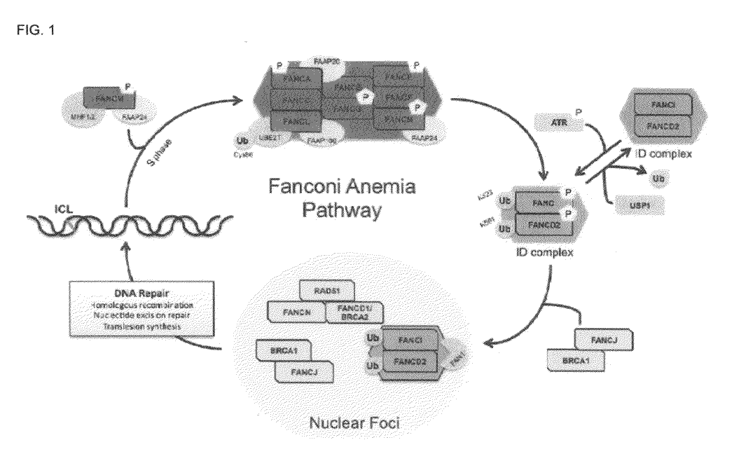

Fanconi anemia (FA), a rare inherited chromosomal-instability autosomal recessive disease, results from mutations in at least one of fifteen genes encoding proteins in the FA pathway shown in FIG. 1. The FA pathway activity is tightly regulated and specifically activated during the S phase of the cell cycle and in response to DNA damage. When DNA is damaged, nuclear FA proteins effect its repair by associating into high molecular weight complexes through a cascade of post-translational modifications and physical interactions. In FA patients, the normal DNA repair pathway is perturbed and proper protein associations do not occur. Mutation of any of the FA genes individually leads to the clinical FA phenotype. FA patient have about a 50-fold increased risk of developing any cancer type, particularly leukemia and SCC. They have shortened life expectancy, birth defects, hematologic abnormalities, and cell and organ hypersensitivity to agents, e.g., mitomycin C, melphalan, diepoxybutane (DEB) that induce DNA interstrand crosslinks (ICL). ICL impair DNA strand separation and unwinding, hindering replication and transcription. Because hematopoietic cells, which are produced in the bone marrow, are particularly sensitive to these protein associations, bone marrow failure (BMF) occurs in most FA patients. Because FA has variable expression of recognized symptoms, the true incidence of FA is likely grossly underestimated.

The importance of the FA pathway, initially regarded as a specialized ICL repair system due to rare incidence of FA, was demonstrated by finding that important breast cancer susceptibility genes BRCA2, PALB2, and BRIP1 are identical to FANCD1, FANCN, and FANCJ. Correspondingly, the FA pathway is activated beyond ICL by forms of genotoxic stress including ultraviolet (UV) and ionizing radiation, and oxidative stress. FA deficient cells are used as a model system to study ATR signaling and BRCA functions in DNA repair.

Carriers of FANCD1/BRCA2 and RAD51C mutations are predisposed to breast and ovarian cancer. Carriers of FANCJ and FANCN mutations are predisposed to breast cancer at lower penetrance. Carriers of FANCD1/BRCA2, FANCC and other FA gene mutations are predisposed to pancreatic cancer. Defects in the FA/BRCA pathway exist in the general, non-FA population, so associated cancer susceptibility is not limited to the rare, inherited scenario.

FA is also characterized by cancer susceptibility, demonstrating that the FA pathway is also important in tumor suppression. FA patient are at risk for developing acute myeloid leukemia (AML). FA patients are at extreme risk for developing squamous cell carcinoma (SCC) of the head and neck (HNSCC), and gynecological tract. The incidence for developing SCC is even greater in bone marrow transplant individuals and individuals recovered from hematopoietic disease.

FA pathway tumor suppression in hematopoietic, versus epithelial, cells could be mechanistically similar or distinct. It is thus important to assess any link between BMF and subsequent solid tumor development, and to optimize therapies to decrease risk of malignant transformation in both hematopoietic and epidermal cells. In one embodiment, leukemia patients who recover poorly from chemotherapy, and young SCC patients, particularly those experiencing serious toxicity from chemotherapy and/or radiation, are candidates for biomarker assessment.

In one embodiment, the inventive method assesses stress responses, DNA repair, and SCC susceptibility.

In the FA pathway, multi-protein interactions coordinate DNA repair. Fifteen complementation groups and corresponding FA genes are known; protein products function as either signal transducers and/or DNA processing factors within the larger FA-BRCA DNA damage response network, subsequently described. The FA/BRCA pathway is activated during DNA replication, and by DNA ICL and other lesion damage. Protein components assemble into at least three complexes within the cell nucleus: FA core complex (FACC), ID complex of (mono)ubiquitinated FANCD2 and FANCI proteins, and a complex of the FANCN and BRCA2 proteins associated with homologous recombination that binds chromatin near DNA lesions downstream from the ID complex.

FACC has eight FA proteins: FANCA, B, C, E, F, G, L, and M, and other FA-associated proteins such as FAAP100, FAAP20 and FAAP24. Each protein is a prerequisite for proper complex assembly and subsequent DNA repair, explaining why mutations in different FA genes yield similar but not necessarily identical clinical phenotypes. FANCM (blue) with MHF1/2 (dark grey) and FAAP24 (light grey) recruit a large multi-subunit ubiquitin E3 ligase, termed FACC (blue), to sites of DNA damage. FACC then mono-ubiquitinates FANCD2 and FANCI; mono-ubiquitinated FANCD2-FANCI (green) is recruited to sites of damage by FANCJ (yellow) and BRCA1 (yellow). These FA proteins colocalize with downstream FA proteins RAD51, FANCN, FANCD1/BRCA2 (yellow), and facilitate DNA ICL repair. Multiple FACC constituents and associated components of the pathway are phosphorylated, including FANCA, G, M, D1/BRCA2, D2, and I; these phosphorylation events are important for ICL repair. FACC forms a nuclear, high molecular weight E3 ubiquitin ligase complex, based on the sole E3 ubiquitin ligase domain of FANCL. FANCL contains additional domains responsible for directing substrate binding (DRWD domain) and four E2 protein interactions (RING domain) by using UBE2T. FANCM provides ICL resistance to cells. FANCM and FAAP24, with the histone-fold containing proteins MHF1 and MHF2, recognize both DNA lesions and stalled replication forks, and subsequently generate single stranded DNA, which is thought to activate both ATR and its downstream target Chk1.

ATR and ATM kinases, key upstream regulators of the FA pathway, phosphorylate several FA pathway components and specifically direct cellular response to DNA damage during S-phase. FANCI phosphorylation likely turns on the FA pathway and initiates interactions between FACC and ID complex (composed of FANCI and FANCD2). Following phosphorylation, FANCI at Lys523 and FANCD2 at Lys562 are monoubiquitinated by FACC ubiquitin ligase, important for downstream function of the FA pathway in DNA repair. FANCD2 mono-ubiquitination is thus widely used as a read-out for FA pathway activation. It is detectable by a shift from the nonubiquitinated short form (FANCD2-S) to a slower migrating, long form (FANCD2-L) in immunoblots. FANCD2-L relocalization to chromatin is visualized using immunofluorescence (IF) by formation of nuclear FANCD2 foci that colocalize with other nuclear DNA damage response proteins (γH2AX or Rad51). Mutation of core complex I components such as FANCA is thus reflected experimentally by the absence of FANCD2 monoubiquitination, and by the absence of FANCD2 and Rad51 foci following DNA damage induction. This has been described for multiple cell types including keratinocytes cultured from FA patient skin biopsies, where retroviral FANCA complementation successfully re-instated detection of FANCD2-L by immunoblot and IF.

ID complex activation initiates its localization to nuclear foci together with BRCA1 and Rad51, essential factors for proper homologous recombination. After ID complex loads onto chromatin, it binds the FAN1 protein, responsible for nuclease activity during repair. Although FANCI monoubiquitination is not required for proper repair, its phosphorylation may play a role in localizing the ID complex to chromatin foci, described as sites of repair because they harbor numerous repair factors such as BRCA1, BRCA2, PCNA, or Rad51. Subsequent DNA nuclease recruitment and activation “unhooks” the crosslink, followed by homologous recombination (HR), nucleotide excision repair (NER) on the opposing strand, and translesion synthesis (TLS) to repair the gap.

Post-DNA repair, the FA pathway is inactivated by de-ubiquitination of the ID complex. Inactivation occurs when USP1, a de-ubiquitinating enzyme, associates with FANCD2 within the nuclear foci and removes the monoubiquitin moiety. The absence of de-ubiquitination of FANCD2, like the absence of ubiquitination, leads to DNA sensitivity to crosslinking agents such as cisplatin or mitomycin C. Although the FA pathway has thus become a model system for ICL repair, its molecular components coordinate and integrate many DNA repair machineries including homologous recombination (HR), nucleotide excision repair (NER), and translesion synthesis (TLS).

In FA patients, clonal evolution plays a key role in progression to leukemia and most FA patients experience progressive BMF and often leukemia. Marrow dysfunction occurs at an early age and is associated with stem cell loss in the hematopoietic compartment, responsible for most FA childhood mortality. The risk of BMF in FA patients is 90% by age 50. Rapid hematopoietic cell loss then forces compensatory chronic proliferation, which likely results in clonal evolution and leukemogenesis. Clonal selection and resistance to cytokine-induced cell death has been reported. In an environment of genomic instability, loss of function of tumor suppressors, as well as oncogenic translocations, can be acquired and selected for rapidly. Chromosomes 1, 3 and 7 were more frequently involved in FA AML than in de novo AML. The specific involved clone may thus distinguish FA AML from de novo cases. The only curative therapy for hematologic FA abnormalities is a bone marrow transplant (BMT). However, genotoxic conditioning regimens and the transplant itself are a significant cause of morbidity and mortality in this patient population, which tolerates chemotherapy and radiation poorly.

FA patients that survive BMF through treatment remain susceptible to cancers, typically myeloid leukemias and SCC. FA patients with biallelic mutations in BRCA2 have an exceptionally high risk of developing AML by age 5, and the overall prevalence of AML in FA patients is 33% by age 40. Sporadic AML may also carry FA defects, but only a small number of mutations have been identified, e.g., FANCF silencing was demonstrated in an AML cell line, and hypermethylation of the promoter regions of FANCC and FANCL was identified in sporadic acute leukemia. The high risk of AML and myelodysplastic syndrome (MDS) in pediatric FA patients, and the similarities between chromosomal abnormalities in FA and AML/MDS suggest molecular link(s) between FA pathway irregularities and AML/MDS in the general population.

Many FA patients develop AML and/or MDS, thus, the FA genotype is viewed as preleukemia. Stabilization and prevention of pre-leukemic phenotypes is the expressed goal, ideally without increasing the risk of solid tumor development. However, a thorough understanding of the role of FA proteins in squamous epithelium is lacking, impeding the rational design of strategies that prevent both hematopoietic and epithelial transformation. Molecular and phenotypic aspects of FA that might differ substantially across tissue and cancer types are presented, and care must be taken to ensure that leukemia prevention and treatment in FA patients does not further exacerbate development of solid tumors.

Mutations in the FA pathway lead to increased risk of SCC, with anogenital region and head and neck SCC (HNSCC) the most common. HNSCC is the sixth leading cancer worldwide, with prolonged tobacco and alcohol use as principal risk factors. In the general population, about 25% of HNSCC are caused by human papillomavirus (HPV) infection, particularly the HPV16 genotype. HPV status determines significant biological differences; HPV positive tumors exhibit improved treatment response and prognosis versus HPV negative tumors. The prevalence of HPV in FA SCC remains controversial.

Because only a few number of individuals exposed to tobacco, alcohol, or HPV ultimately develop cancer, one who does must have an inherent predisposition that collaborates with these genotoxic exposures for cancer to occur. Within the general population there are different levels of DNA safeguarding capability, which play a role in cancer development. Unfortunately, overall treatment outcomes for HNC have not improved, and conventional clastogenic therapies have substantial side effects on normal physiological functions, e.g., swallowing, speech, and physical appearance. These responses to therapy, together with tumor development, are also modified by genetic predispositions, and appear to be greatly amplified in FA patients. Thus, while FA tumors are predicted to be exquisitely sensitive to conventional chemotherapy and radiation, the FA patient's global hypersensitivity to DNA damage limits their effectiveness, particularly radiation therapy.

Array based comparative genome hybridization of 21 sporadic oral SCC revealed deregulation of FA and FA-associated genes, including BRCA1, BRCA2 (FANCD1), FANCG and FANCD2. Table 1 lists in either FA genes themselves, or in a select gene subset associated with DNA repair. Table 1 shows somatic mutations of HNSCC patient tumors in a select subset of FA- and DNA-repair associated genes, each letter in parentheses represents each patient. Mutations were identified by exome sequencing of tumors isolated from sporadic HNSCC patients. Table 1 represents new analyses previous data that revealed 38/74 (51%) sequenced tumors harbored at least one somatic mutations in the indicated gene subset. A majority of these tumors (18/38=47%) harbored multiple mutations, ranging from 2-7. Among these mutated genes, BRCA2, FANCM (5 mutations reported), ATR, UBE4A (4 mutations reported), BRCA1, USP43 and USP44 (3 mutations reported) are most frequent. These findings indicate a subset of primary, therapy-naïve HNSCC harbor mutations in important DNA repair pathways including FA. Such mutations may contribute functionally to increased tumor growth and/or may modify sensitivity to conventional drug therapies.

TABLE 1

| FA-Associated | | Mutation | Amino Acid |
|---|---|---|---|
| Genes | Description | Types | Change |
| ATM | ataxia telangiectasia mutated isoform 1 | Missense | p.I2899M |
| | | Missense | p.S974F^{(a)} |
| ATR | ataxia telangiectasia and Rad3 related protein | Nonsense | p.W1784*^{(b)} |
| | | Missense | p.S1701F^{(b)} |
| | | Missense | p.E1840Q^{(c)} |
| | | Missense | p.A248S^{(c)} |
| BRCA1 | breast cancer 1, early onset isoform 2 | Missense | p.E554G |
| | | Missense | p.R1670K^{(d)} |
| | | Missense | p.V6271^{(d)} |
| BRCA2 | breast cancer 2, early onset | Nonsense | p.Y2884* |
| | | Missense | p.A1411T |
| | | Missense | p.E97V^{(e)} |
| | | Splice_Site | ^{(f)} |
| | | Missense | p.K1530E |
| BLM | Bloom syndrome protein | Missense | p.D294N^{(g)} |
| ERCC5 | XPG-complementing protein | Missense | p.Q1002R^{(a)} |
| ERCC6 | excision repair cross-complementing rodent | Missense | p.R928T^{(h)} |
| ERCC8 | excision repair cross-complementing rodent | Missense | p.D371H^{(t)} |
| FANCC | Fanconi anemia, complementation group C | Missense | p.T319R^{(b)} |
| FANCI | Fanconi anemia, complementation group | IMissense | p.C558F |
| FANCM | Fanconi anemia, complementation group M | Missense | p.I633M^{(l)} |
| | | Missense | p.V1951E^{(a)} |
| | | Missense | p.H2016D^{(e)} |
| | | Missense | p.S981L^{(k)} |
| | | Nonsense | p.Q1701* |
| RAD50 | RADSO homolog isoform 1 | Missense | p.E1106Q^{(e)} |
| RAD51AP1 | RAD51 associated protein 1isoform a | Missense | p.S263C |
| RAD51AP2 | RAD51 associated protein 2 | Nonsense | p.QS*^{(d)} |
| | | Missense | p.S355R |
| RAD51C | RAD51 homolog C isoform 1 | Missense | p.M1181^{(l)} |
| | | Missense | p.Q340K^{(m)} |
| RAD51L1 | RAD51-like 1 isoform 2 | Missense | p.E167K^{(g)} |
| RAD52 | RAD52 homolog | Missense | p.G59R⁽ⁿ⁾ |
| RAD54B | RAD54 homolog B | Missense | p.L428V⁽ⁱ⁾ |
| RAD9B | RAD9 homolog B | Frame_Shift | p.V412fs^{(h)} |
| RADIL | Rap GTPase interactor | Missense | p.P249R^{(o)} |
| REV1 | REV1-like isoform 1 | Missense | p.R874W^{(p)} |
| RMI1 | RMI1, RecQ mediated genome instability 1, | Missense | p.E617D^{(m)} |
| UBE2E2 | ubiquitin-conjugating enzyme E2E 2 | Missense | p.S46F^{(h)} |
| | | Missense | p.D22H^{(g)} |
| UBE2I | ubiquitin-conjugating enzyme E21 | Missense | p.W103C⁽ⁱ⁾ |
| UBE2J2 | ubiquitin conjugating enzyme E2, J2 isoform1 | Missense | p.I113F^{(j)} |
| UBE2NL | ubiquitin-conjugating enzyme E2N-like | Nonsense | p.K95*^{(r)} |
| UBE2Q1 | ubiquitin-conjugating enzyme E2Q | Missense | p.M2061^{(k)} |
| UBE3A | ubiquitin protein ligase E3A isoform 2 | Missense | p.K489E^{(a)} |
| | | Missense | p.S166R |
| UBE4A | ubiquitination factor E4A | Missense | p.E616K |
| | | Missense | p.E822D⁽ⁱ⁾ |
| | | Missense | p.E827K⁽ⁱ⁾ |
| | | Missense | p.LGOSV |
| UBE48 | ubiquitination factor E4B isoform 1 | Nonsense | p.E114*^{(o)} |
| USP12 | ubiquitin thiolesterase 12 | Missense | p.A341E^{(j)} |
| USP13 | ubiquitin thiolesterase 13 | Missense | p.ASGIT^{(s)} |
| | | Missense | p.P729L^{(d)} |
| USP19 | ubiquitin thioesterase 19 | Nonsense | p.Y941* |
| USP40 | ubiquitin thioesterase 40 | Nonsense | p.G641*^{(o)} |
| USP44 | ubiquitin thioesterase 44 | Missense | p.E226Q⁽ⁿ⁾ |
| | | Missense | p.E167Q⁽ⁿ⁾ |
| | | Missense | p.S64T⁽ⁿ⁾ |
| USP7 | ubiquitin specific peptidase 7 | Missense | p.M1070V |
| USPS | ubiquitin specific peptidase 8 | Missense | p.D359H^{(t)} |
| | | Missense | p.V736I |
| USP29 | ubiquitin specific peptidase 29 | Missense | p.T584N^{(r)} |
| USP36 | ubiquitin specific peptidase 36 | Missense | p.I191M^{(k)} |
| USP45 | ubiquitin specific peptidase 45 | Missense | p.K180R^{(t)} |
| USP17L2 | deubiquitinating enzyme 3 | Missense | p.Q430E^{(k)} |
| USP4 | ubiquitin specific protease 4 isoform a | Missense | p.R179P^{(t)} |
| | | Missense | p.R40Q^{(f)} |
| USP24 | ubiquitin specific protease 24 | Missense | p.Y343H^{(c)} |
| USP26 | ubiquitin-specific protease 26 | Missense | p.K734N |
| | | Missense | p.V906L^{(p)} |
| USP28 | ubiquitin specific protease 28 | Missense | p.E371Q^{(k)} |
| USP35 | ubiquitin specific protease 35 | Missense | p.E55K^{(k)} |
| USP43 | ubiquitin specific protease 43 | Missense | p.S455F |
| | | Missense | p.R206Q^{(k)} |
| | | Missense | p.E834V^{(t)} |
| USP47 | ubiquitin specific protease 47 | Missense | p.I1263V |
| USP51 | ubiquitin specific protease 51 | Missense | p.F624L^{(j)} |
| USP9X | ubiquitin specific protease 9, X-linked isoform | Missense | p.P1105L^{(s)} |
| | | Missense | p.W512L^{(P)} |
| USP9Y | ubiquitin specific protease 9, V-linked | Missense | p.1226F |
| WDR48 | WD repeat domain 48 | Splice_Site | ^{(l)} |

In contrast to leukemogenesis, solid tumor development, and particularly HNSCC and anogenital SCC, occur early and with striking aggressiveness in FA patient. Surgical treatment remains the mainstay, but relapse-free, two-year survival rates are <50%. Chemotherapy and radiation treatments are associated with high mortality and morbidity due to FA patients' global hypersensitivity to DNA damage; complications from radiotherapy include mucositis and pancytopenia.

New therapies for clinical management of FA associated SCC are thus needed. Early intervention, the best line of defense, is key for increasing the chance of complete surgical resection and survival. The previous absence, until now, of relevant biomarkers impeded early detection of tumor cells in FA patients.

Hematopoietic stem cells (HSC) in FA patient, unlike normal stem cells, are hypersensitive to apoptosis-inducing cytokines such as tumor necrosis factor-alpha (TNF-alpha). Exposure to these cytokines reduces the number of FA HSC by increasing the apoptosis rate in these cells. These events lead to rapid bone marrow failure with decreased production of all differentiated cell types. FA HSC adapt to this hostile environment by developing mutations, which confer resistance to TNF-alpha but retain the characteristic sensitivity to crosslinkers. Reducing the selection pressure in FA patients in order to decrease the transformation of FA hematopoietic cells towards leukemia is important.

Patients with sporadic HNSCC frequently present at advanced disease stages because of inferior diagnostic approaches. This necessitates aggressive chemo- and/or radiation therapy, leading to extreme toxicity and sometimes death. Over 20% of FA patients diagnosed with a solid tumor had a FA diagnosis made only after cancer was detected, thus treatment toxicity was not predictable. There is thus an urgent need for improved, early diagnosis of HNSCC in FA patients, and also for a FA diagnosis prior to cancer development and treatment. Cancer prevention in this patient population should be the primary goal.

Previous approaches to identifying markers of sporadic HNSCC are restricted to global genomic and proteomic approaches. Metagenomics, a specific type of genomic analysis, isolates nucleic acids and performs unprejudiced DNA sequencing to categorize viruses and microorganisms present in a particular sample. Mass spectrometry (MS) determined changes in proteins M2BP, MRP14, and CD59, validated by immunoassays. Proteome/peptidome analysis and matrix-assisted laser desorption/ionization-time of flight-mass spectrometry (MALDI-TOF-MS) was used, but requires optimization to establish practicality in early stage detection in a clinical setting, with time and cost also being factors. Such “omics” techniques have identified several potential biomarkers of head and neck cancer, but additional techniques will be beneficial. For example, results from these “omic” methods may not fully correlate to one another because regulation of certain genes does not always result in abundance in the corresponding proteins. This is not surprising, since the production of a protein may occur sometime after its gene is transcriptionally activated and indicates increased emphasis on protein expression levels within a given system. Proteins reflect biological processes that take place at a defined time in a certain system, and metabonomics is the next most accurate technology for observing intermediates and final products relating to both gene transcription and protein regulation.

Metabonomics is the study of an organism's metabolome: quantitative measurements of multiparametric metabolic response to pathophysiological stimuli or genetic modifications. It has minimal use to date in cancer biology, but is potentially useful for biomarker identification in disease because the metabolome is downstream of both the genome and proteome and is complementary to other “omic” methods. Metabonomics research in HNSCC should address normal metabolic function in epidermal compartments to define how changes in this metabolic fingerprint can function as early diagnostic tools. Defining changes in the metabolome related to disease onset and progression in the FA patient HNSCC provides information about regulatory pathways in cancer in general, and will drive biomarkers identification and therapeutic targets

Thus, methods for diagnosing FA and for adjusting or modifying cancer treatment in a FA patient, based on the diagnosis of FA, is needed and biomarkers for FA were identified.

In one embodiment, biomarkers were identified using NMR-based metabonomics in biological samples from FA patients and FA knockout mice. FA knockout mice exist for various components of the FA pathway (Bakker et al. Learning from a paradox: recent insights into Fanconi anaemia through studying mouse models. Dis Model Meh. 6 (2013) 40). However, these mice do not spontaneously mimic characteristic FA manifestations, e.g., anemia, leukemia, and solid tumor development. Based on variable expressivity of recognized symptoms, and also on lack of biomarkers, the true incidence of FA is likely grossly underestimated. The disclosed biomarkers will assist screening of susceptible patients such as leukemia patients who recover poorly from chemotherapy, and young patients with SCC or other solid tumors, particularly those who experience serious toxicity from chemotherapy and/or radiation.

Current assays used for clinical diagnosis of FA are highly specialized, time consuming, and expensive. Further, there are no biomarkers available for the diagnosis of many other DNA repair deficiencies.

One embodiment is a method for diagnosing individuals in the FA population and on a therapy regimen, especially cancer patients and especially HNSCC patients and patients after BMF, at the earliest possible stage, and then modifying the current therapy as needed, based on the biomarker and or biomarker signature level, to improve outcomes. Improved outcomes include, but are not limited to, improved treatment response, increased or decreased treatment dose, increased or decreased treatment frequency, different treatment types, removal of one treatment type, substitution of another treatment type, addition of another treatment type, etc.

One embodiment used nuclear magnetic resonance (NMR) metabonomics to identify a unique metabolic biomarker signature for FA in both mice and human biological samples. The method is innovative and clinically relevant, because the results provide an otherwise poorly understood link between metabolic response and DNA repair and provide for development of NMR spectroscopy as a diagnostic and surveillance tool in both children and adult FA patients. These data advance a mechanistic understanding of how metabolism marks or is functionally associated with DNA deficiency syndromes including FA.

One embodiment uses NMR with or without more routine clinical assays to detect and quantitate the biomarker or biomarker signature. Clinical chemistry assays for the disclosed biomarkers are known in the art, e.g., Burtis, Ashwood, and Bruns, Tietz Fundamentals of Clinical Chemistry (2007) 6th Ed., Saunders for assaying 3-hydroxyisovalerate, acetate, isopropanol, glycerol-3-phosphocholine, choline, histamine, glutamate, alanine, glycine, isoleucine, lysine, phenylalanine, threonine, tyrosine, vitamin A, vitamin D, cholesterol, ketone bodies, acetone, and/or acetoacetate/acetoacetaldehyde.

The methods include assessment whether an individual has or is predisposed to developing FA for making a change in treatment. For example, and without limitation, any form of fluid assay capable of detecting biomarkers may be used. The assay may be performed in a clinical laboratory or may be performed at a point of care and includes kit assays. A kit includes instructions for use and reagents for performing the assay. It may include a test substrate, e.g., test strip, dip stick, cassette, cartridge, panel, chip-based or bead-based array, multi-well plate, or series of containers, etc. The assay may be qualitative (present or absent), quantitative (concentration), or semi-quantitative (high or low relative to a control or to a predetermined threshold quantity). One or more reagents is provided in or on the test substrate to detect the presence (qualitative), concentration (quantitative), and/or relative amount compared to a control (semi-quantities) of one or more of the disclosed biomarkers. The kit may include assay panels for a number of biomarkers, with reagents provided at predetermined locations configured on separate test strips, or sequentially or in tandem on one test strip, and may include positive and/or negative controls on the same or different test strips. The biological sample may be dispensed directly or indirectly to contact the reagent(s), and the sample may be directly from the patient or previously obtained. The results may read by any of chromogenic, fluorogenic, electrochemiluminescent, etc. The assay may be an enzyme immunoassay (EIA), e.g., an enzyme-linked immunoassay (ELISA).

As an illustrative example only, a chip contains reagents immobilized at discrete, predetermined locations for detecting and quantitating in a fluid sample the concentration of the biomarkers 3-hydroxyisovalerate, acetate, isopropanol, glycerol-3-phosphocholine, choline, and/or histamine. The chip may be configured such that a detectable output, e.g. color change, is provided only if the concentration of one or more of these biomarkers exceeds a threshold value selected to distinguish between a biomarker concentration indicative of a healthy individual versus an individual having or predisposed to developing FA. Thus, the presence of a detectable output such as a color change provides an immediate indication that the sample contains significantly different concentrations of one or more biomarkers compared to a control, indicating that the subject has or is predisposed to developing FA and indicating that therapy should be altered.

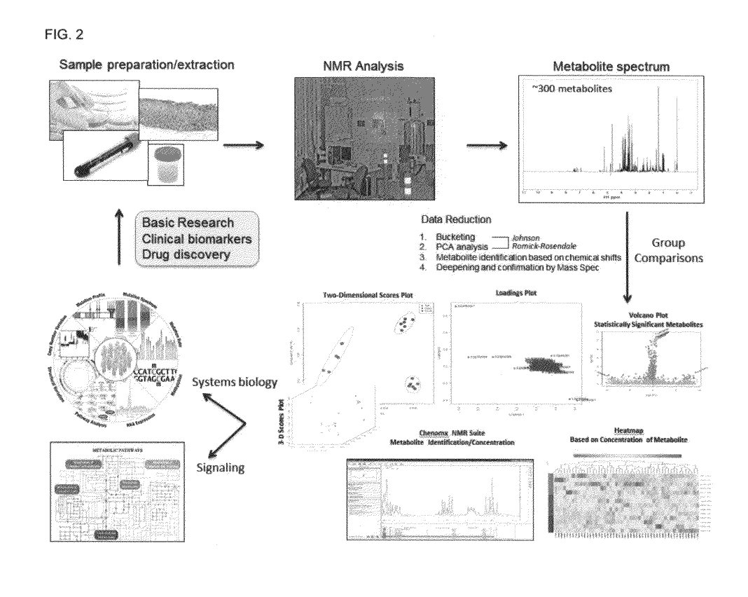

FIG. 2 outlines the steps involved in one embodiment of the metabonomic method. It starts with sample preparation/extraction, then proceeds to nuclear magnetic resonance (NMR) analysis resulting in a metabolite spectrum, then group comparisons using volume plot of statistically significant metabolites, loadings plot, and a two-dimension scores plot. A heatmap based on metabolite concentration, and an NMR suite of metabolite identification/concentration can be used for both signaling and systems biology for clinical biomarker and drug target identification.

Biological fluids and tissues, and cell culture extracts, were collected from mice and humans, and processed prior to analyses. Biological samples composed of cells obtained from culture plates, blood plasma, and urine were either concentrated using a SpeedVac centrifuge or a lyophilizer. The dried samples were then resuspended in a phosphate buffer containing both D₂O and trimethylsylilpropionate (TMSP), an internal standard used for quantification of metabolite concentrations. Biological samples were then transferred to an NMR tube prior to analysis. All samples were analyzed on a 600 MHz NMR spectrometer. Spectral data were processed by data reduction software and analyzed by multivariate statistical analysis software that identified unique metabolic signatures compared to either isogenic FA-proficient cells or to healthy control subjects. These results may be further analyzed for biological relevance and linked to metabolic disease states, signaling pathways and other “omics” data. The results demonstrated the feasibility of using NMR-based metabonomics to identify clinical disease marker, assess drug or other therapy of a patient being treated for a disease, provide modifications to existing therapy and/or identify alternate drug or other therapies, in addition to addressing basic research questions.

In one embodiment, an FA-specific metabolic signature was identified in biological samples from a FA patient and from FA-knockout mice. The metabolic signature or pattern differs from that of family members for patients, and from littermates for mice.

Epithelial tissues, hematopoietic stem cells, serum, urine, and feces from FANCA, FANCC and FANCD2 knockout mice, and from their wild type and heterozygous littermates, are analyzed by NMR spectroscopy. The resulting NMR profiles provide the metabolic fingerprint of each murine specimen, and metabolites that are specific to FA knockout mice are identified using multivariate statistical significance analysis and existing small metabolite databases. Metabolic profiles of FANCA, FANCC and FANCD2 knockout mice are also compared to one another and unique biomarkers specific to each complementation group are identified. This is significant because distinct complementation groups in humans vary in disease severity and risk of cancer, but a diagnosis of complementation group is currently expensive and inconclusive in 20% of cases at the FA Comprehensive Care Center at Cincinnati Children's Hospital.

Urine, skin and blood from FA patients and their family members were analyzed by NMR spectroscopy. Unique metabolic signatures for both FA and specific complementation groups, where known, were identified. Over fifty urine samples from FA patients and their family members were collected, and urine samples from normal, age-matched controls, i.e., individuals who did not have FA, were obtained. Levels of acetone and acetaldehyde in the urine were a distinguishing feature of FA.

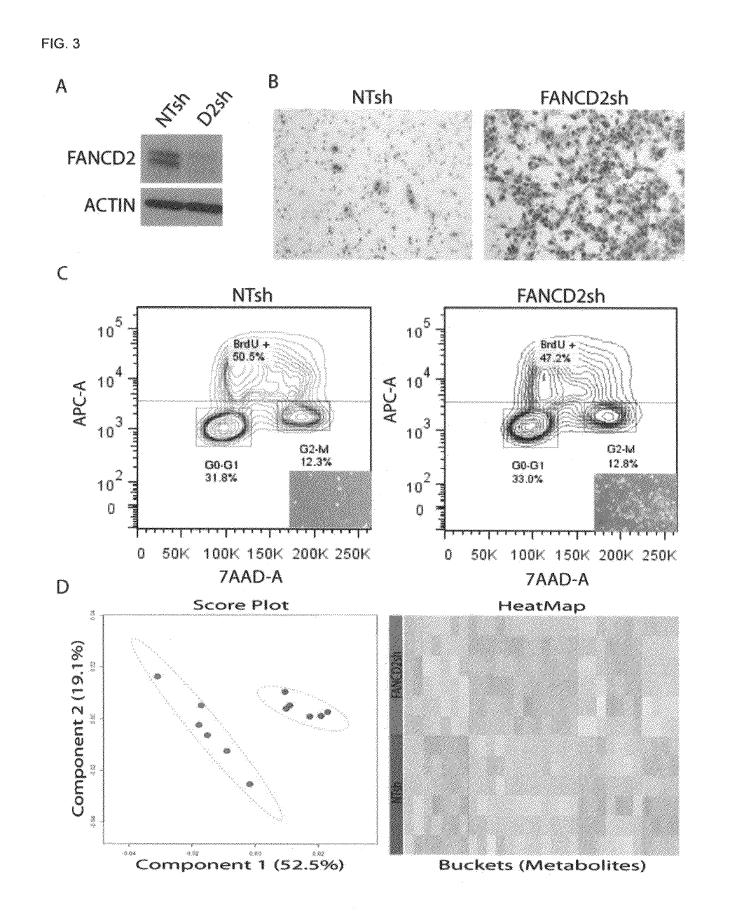

Metabonomic and RNA sequencing studies were performed with (i) immortalized keratinocytes or SCC cells isolated from FA patients, either control transduced or corrected for the appropriate FA gene using retroviral transduction, i.e., the gene mutation was identified and the non-mutated functional gene was added to restore FA pathway activity (data not shown), and with (ii) SCC cells cultured from tumors of individuals with sporadic HNSCC that were knocked down for a key component and central regulator of the pathway FANCD2. Results are shown in FIG. 3. FIG. 3A is a Western blot showing knockdown of FANCD2 (FANCD2sh) in a sporadic HNSCC cell line compared to non-targeting shRNA (NTsh) control. FIG. 3B shows an invasion assay, demonstrating that FA loss alters tumor biology, e.g., stimulates metastatic potential in addition to altered DNA repair, and showing increased invasion in FANCD2-deficient cancer cells. FIG. 3C shows similar cell cycle profiles of FA-proficient (NTsh) and FA-deficient cells. FIG. 3D left is a score plot showing that the metabolic profiles of cell extracts from NTsh and FANCD2sh cells are uniquely distinct from one another. FIG. 3D right is a heat map showing statistically significant changes in concentration of a number of metabolites within each cell type.

Functional complementation and knockdown were verified in each case using methods known in the art. The FA status did not affect cellular proliferation, as shown in FIG. 3C. FANCD2 loss dramatically stimulated cellular invasion, as shown in FIG. 3B, a novel tumor phenotype for FA deficient epithelial cells that might contribute to the strikingly aggressive course of FA tumors in patients, and is likely independent from the well characterized DNA repair defects in such cells. Metabonomics data obtained from these same cells showed that distinct patterns of small metabolites distinguish isogenic FA-proficient cells from their FA-deficient counterparts. FIG. 3D demonstrates metabolic differences between HNSCC cells that either have a functional FA DNA repair pathway (green) or have an inactivation of the pathway (red) due to knockdown of FANCD2. The FIG. 3D score plot is a two-dimensional representation of the separation between the groups, and each point on the plot represents a separate cellular NMR spectrum. The FIG. 3D heat map shows the differences in intensities, i.e., concentrations of the buckets (metabolites) between the FA-proficient (NTsh) and FA-deficient (FANCD2sh) cells. Similar metabolic differences were observed in the above FA patient cells compared to their complemented counterparts (data not shown). The data demonstrated the disclosed method of using small metabolites in biological samples from FA patients and FA knockout mice are useful to rapidly diagnose FA as a genetic syndrome, and that specific metabolites are biomarkers for SCC in FA patients and beyond.

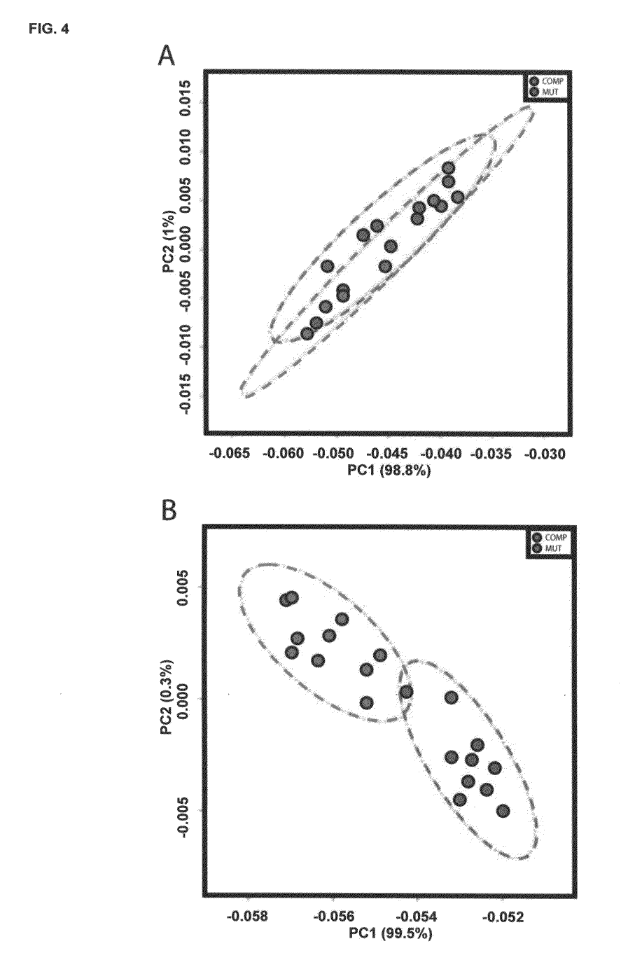

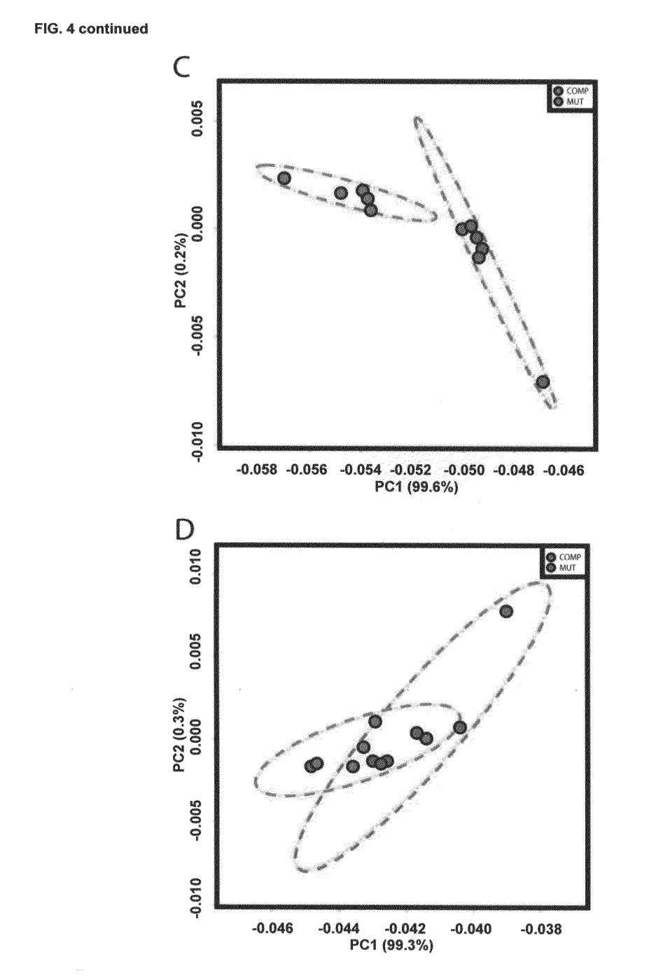

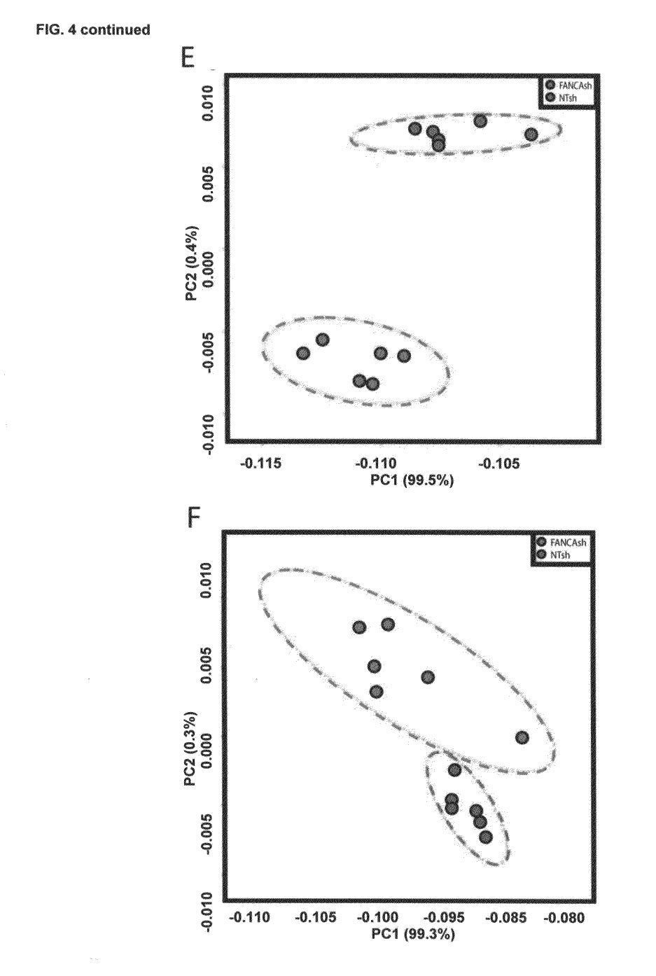

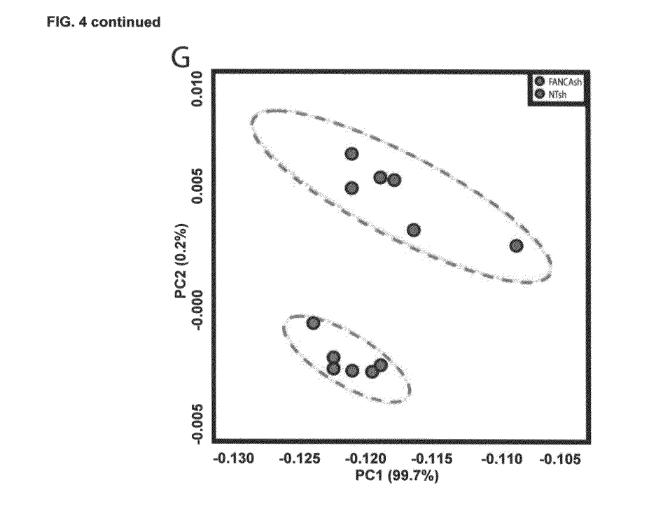

Metabolic profiling analysis was conducted in seven cell lines, each either expressing or with a knockdown of FANCA. The results are shown in FIG. 4, with each of FIGS. 4 A, B, C, D, E, F, and G showing results of a separate cell line. The Score plot is a two-dimensional representation of the differences (separation) observed between the FA-proficient control and the FA-deficient groups. For example, FIG. 4A compares cells that harbor a mutation in the FANCA gene (green) and those that have been complemented or the functional FANCA gene has been added back into the cells (red). Each point on the Score plot represents a separate NMR spectrum.

The results are summarized below:

| | Mahalanobis | | | | |
|---|---|---|---|---|---|
| | Distance | Two-sample | | Criitcal | |
| Cell Lines | (MD) | T2 statistic | F-value | F-value | Significance |
| IHK-1FA | 1.21 | 6.18 | 2.88 | 3.73 | No |
| IHK-2FA | 4.48 | 100.15 | 47.29 | 3.18 | Yes |
| HNc-1FA | 4.29 | 50.25 | 22.33 | 6.26 | Yes |
| HNc-2FA | 1.70 | 8.65 | 3.89 | 5.05 | No |
| HNc-1 | 17.50 | 918.56 | 413.35 | 5.05 | Yes |
| HNc-2 | 5.02 | 75.55 | 34.00 | 5.05 | Yes |
| HNc-3 | 12.16 | 443.45 | 99.55 | 5.05 | Yes |

The Mahalanobis Distance (MD) is the distance between the centroids of each of the study groups. The distance is then determined to be statistically significant by determination of the T2 value and F-value. The experimental F-value is then compared to the Critical F-value and deemed significant if the experimental F-value is larger than the Critical F-value.

As described above, a unique metabolic signature distinguishes FA-proficient, meaning a FA pathway with no mutations or silencing events, and FA-deficient, meaning at least one gene in the FA pathway is mutated or silenced, cancer cells (FIG. 3D). Such FA deficiency dramatically increases tumor invasiveness but not tumor proliferation in vitro (FIGS. 3B, 3C).

Thus, FA deficiency in the germline, or somatically in the general population, may stimulate tumor cell invasion and metastasis in vivo, as will be assessed by results of the growth of isogenic, FA-proficient versus FA-deficient cells in the flanks and tongues of immunodeficient mice.

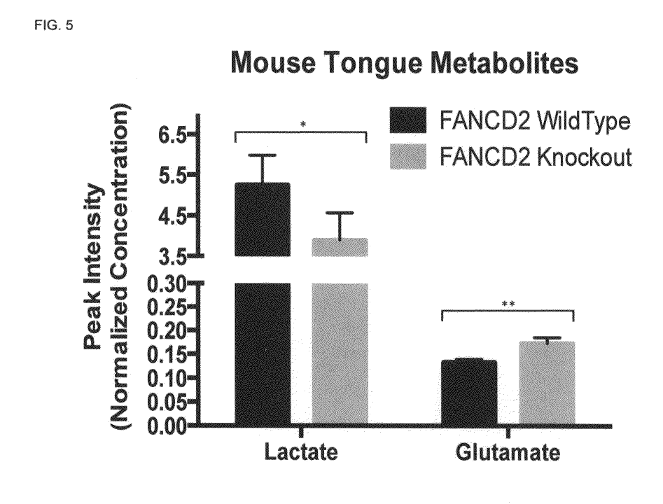

Non-tumorigenic FANCD2 positive and FANCD2 negative tongues of mice were used for NMR. The results, shown in FIG. 5, indicated that transformed FA SCC mice had measurable metabolic differences compared to wild type FANCD2 mice. Metabolic profiling of FANCD2 knockout mice, compared to FANCS2 wild type mice, had a significant decrease in the biomarker lactate and had a significant increase in the biomarker glutamate in normal tongue tissue.

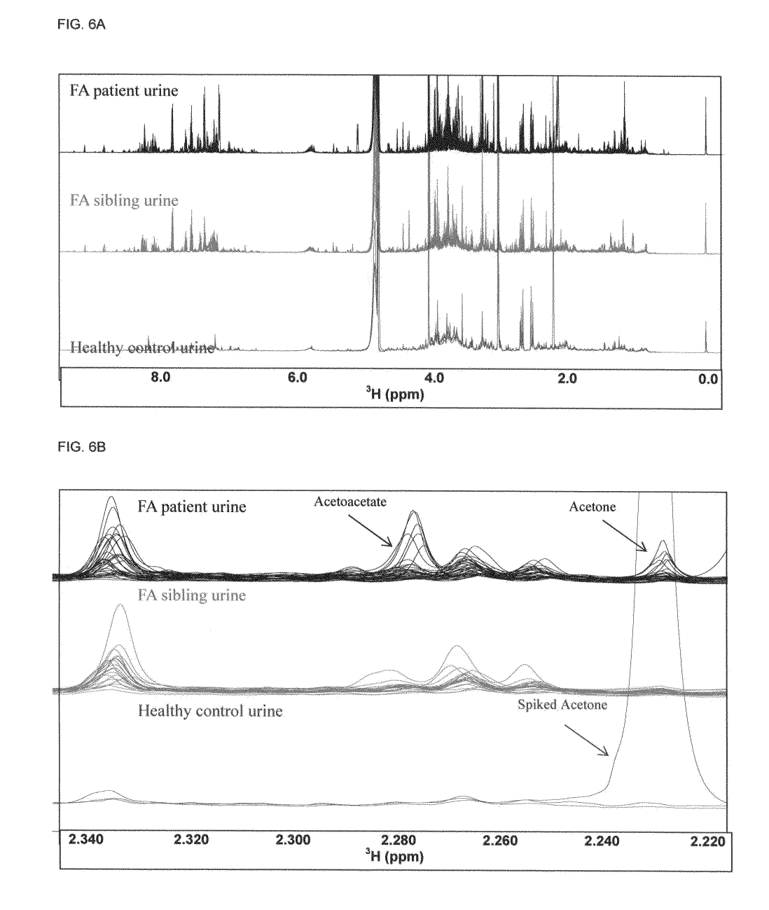

The method permits identification of carriers of FA gene mutations who are themselves unaffected may be indentified using the inventive method, as shown by results in FIGS. 6A and 6B. Carriers of FA, who are unaffected, cannot currently be indentified. Such indentification is needed to provide more information, e.g., more informed pregnancies, early care for newborns, genetic counseling, pre- and post-implantation screens of embryos, etc. The method provides hippurate and phenylalanine as biomarkers for identification of carriers of FA gene mutations.

First morning urine was collected from individuals with FA, their siblings, and unrelated, healthy controls. Aliquots of the specimens were frozen on dry ice, then thawed on ice. A 630 μL volume of urine was added to 70 μL phosphate buffer containing 100% D₂O and trimethylsilylpropionate (TMSP), resulting in a final mixture of urine, buffer, 10% D₂O and 0.58 mM TMSP. A 600 μL aliquot of each mixture was analyzed on a 600 MHz Varian NMR. Results are shown in FIG. 6A.

Analysis of urine from 33 FA patients, 20 unaffected siblings which are often heterozygous for FA, and two healthy control subjects revealed elevated levels of acetone and acetoacetate in FA patients compared to their siblings and healthy unrelated controls. FIG. 6B shows a zoomed-in spectral image of FIG. 6A showing the region encompassing both the acetone and acetoacetate metabolite peaks, with the acetone spiked healthy control sample indicated. The acetone metabolite peak was confirmed by spiking one of the healthy control urine samples. There were increased concentrations of metabolite peaks in the aromatic region (5 ppm-10 ppm) of the spectrum where both hippurate and phenylalanine reside; the overall majority of urine metabolic profiles within the three groups was similar. Elevated hippurate and phenylalanine levels were detected in both FA and sibling populations when compared to healthy control subjects.

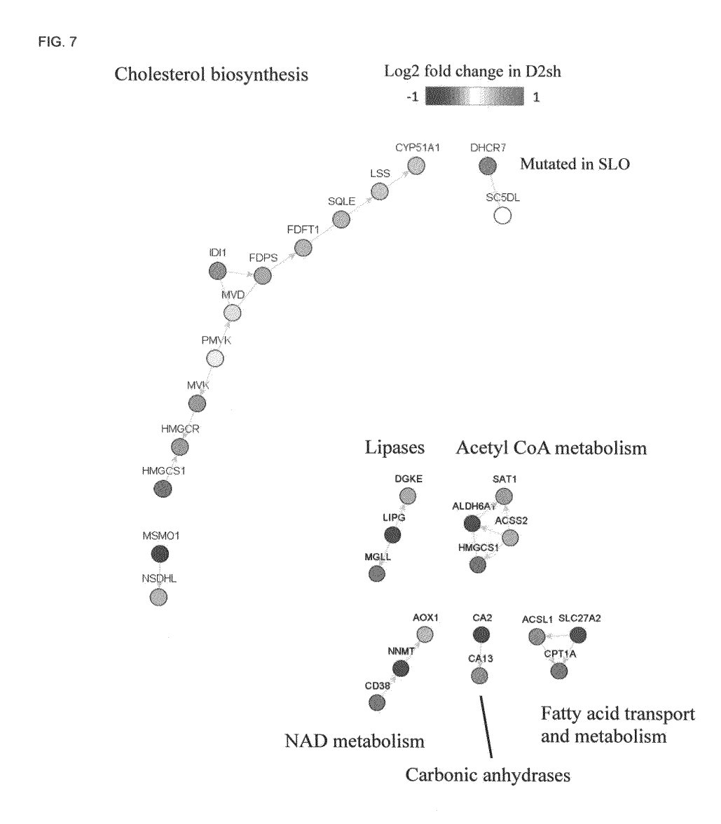

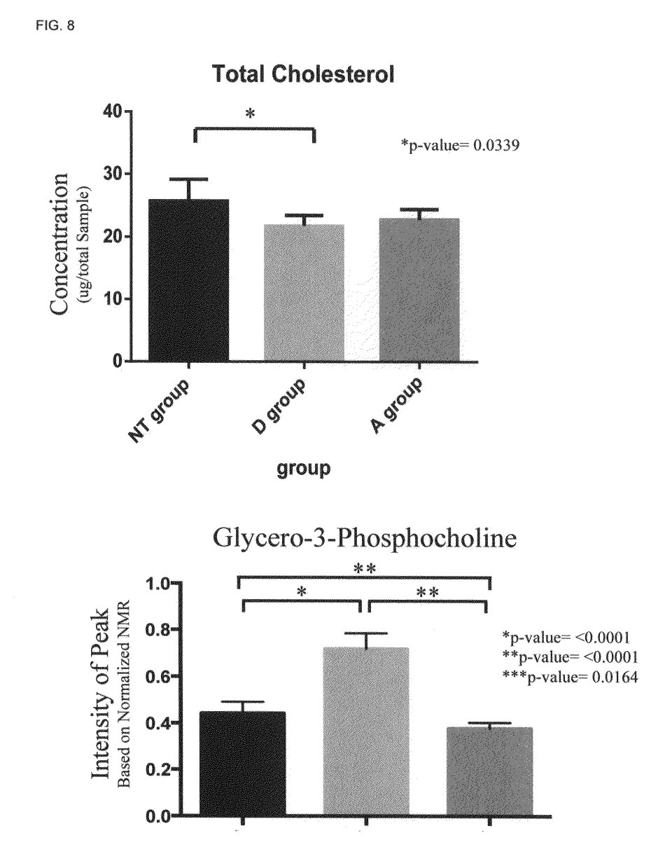

A subset of the FA-proficient and FA-deficient tumors was subjected to RNASeq analysis to determine transcriptional patterns that correlate with the observed metabolic and invasive changes. The results indicated that FA de-regulated cellular metabolism and decreased expression of enzymes involved with lipid metabolism. FIG. 7 shows that lipid metabolism gene expression was attenuated in FANCD2sh SCC1 cells (FIG. 7). In one embodiment, the disclosed metabolite FA biomarkers can include genetic biomarkers. In one embodiment, the genetic biomarkers may be used without the metabolite FA biomarkers. It has also been discovered that cholesterol levels are low in FANCD2sh keratinocytes (FIG. 8A). NT group is control cells transduced with non-targeting shRNA expression vector. D group is knockdown cells transduced with FANCD2 shRNA. A group is knockdown cells transduced with FANCA shRNA. Use of knockdown expression vectors, such as in D group and A group results in inactivation of the FA pathway which mimics those cells harboring FA gene mutations as seen in patient samples. FIG. 8B shows levels of the biomarker glycerol-3-phosphocholine using the same NT, D, and A groups as in FIG. 8A.

The results demonstrated the utility of the disclosed metabolites as biomarkers of FA, FA-deficient tumors, and/or BMF. The method is minimally invasive and provides results within a short time from sample collection to biomarker level determination in the patient compared to controls. Biological samples are those routinely clinically assayed, e.g., urine, plasma, saliva. In one embodiment, biomarker levels in patient saliva samples are compared to biomarker levels in patient related family member saliva samples. Saliva is relatively easily obtained, and salivary metabolic data may be related to urine data, and may be ultimately integrated with the FA SCC metabolome to provide clinical information on the presence of pre-malignant and malignant lesions in those same patients.

The disclosed NMR-based metabonomics revealed unique small metabolite signatures in FA cells, e.g., as shown in FIG. 3D. The metabolites exhibiting different levels were identified. In one embodiment, an altered patient level of at least one of 3-hydroxyisovalerate, acetate, isopropanol, glycerol-3-phosphocholine, choline, histamine, glutamate, alanine, glycine, isoleucine, lysine, phenylalanine, threonine, and/or tyrosine, compared to a control level, was used to diagnose FA. In one embodiment, an increased level of at least one of 3-hydroxyisovalerate, acetate, isopropanol, glycerol-3-phosphocholine, choline, and/or histamine in a patient compared to a control indicates FA. In one embodiment, a decreased level of at least one of glutamate, alanine, glycine, isoleucine, lysine, phenylalanine, threonine, and/or tyrosine in a patient compared to a control indicates FA. In one embodiment, an assay FA signature or panel permits enhanced determination when the level of more than one biomarker is altered in the patient compared to the level of those same biomarkers in the control. In embodiments, the FA signature or panel measures biomarkers that increase with FA, measures biomarkers that decrease with FA, measures biomarkers that increase and decrease with FA, and includes control levels of biomarkers. Control biomarkers can be used to control for sample variations, such as amount or concentration.

Biological samples are obtained from patients and control by known protocols. In embodiments, the controls are matched by at least one characteristic including gender, age, genetic relative, etc. Samples may be obtained during routine screening or in connection with a regular check-up or physician visit, or together with administration of treatment such chemotherapy, radiation therapy, surgical therapy, etc.

In one embodiment, the patient is stressed prior to obtaining the biological sample for analysis. For example, one biomarker panel includes components involved with lipid metabolism, such as cholesterol metabolism. The patient thus may be subjected to dietary restrictions, e.g., instructed to fast for 12-18 hours, prior to sample collection to induce a state of ketosis or decreased cholesterol. Decreased cholesterol levels, i.e., hypocholesterolemia, is seen in Smith-Lemli-Opitz (SLO) syndrome, which is the result of a mutation in 7-dehydrocholesterol reductase (DHCR7). DHCR7 mRNA is decreased in FA cells and produces FA-like signs and symptoms such as developmental disorder, organ malformation including genitalia, distinctive facial features, microcephaly, intellectual disability, failure to thrive. Cellular and animal models of SLO demonstrate oxidative stress. Lack of cholesterol is associated with insufficient bile acid, taurine, in the gut and fewer micelles for vitamin and fat absorption leading to measurable vitamin deficiency. Thus, in one embodiment, FA biomarkers include cholesterol, vitamin A, and vitamin D. Because steroid hormones are generally synthesized from cholesterol, the reduction in cholesterol will likely result in decreased steroid hormone levels. Thus, in one embodiment, FA biomarkers include steroid hormones. Examples of steroid hormones are known in the art and include glucocorticoids, mineralocorticoids, androgens, estrogens, progestogens, and the closely related Vitamin D compounds.

Samples can be collected one or more times for a separate or combined analysis. The collected samples may be assayed immediately or stored under appropriate conditions for subsequent analysis. Analytical methods for bio marker level determination can include one or a combination of mass spectrometry (MS) coupled with gas chromatography (GCMS) or liquid chromatography (LCMS), HPLC, NMR spectroscopy, TLC, electrochemical analysis, refractive index spectroscopy, ultra-violet spectroscopy, fluorescent analysis, radiochemical analysis, near-infrared spectroscopy and light scattering analysis. In some embodiments, fluid samples may be processed prior to analysis. Biomarker quantitation may be performed using tools that compare the integral of a known reference signal with signals derived from a biomarker to determine concentration relative to the reference signal. Levels of the specific biomarker over or below a determined critical value, either in concentration or in amount, can indicate the presence of FA. The biomarker(s) concentrations or levels can be interpreted independently using an individual cut-off for each biomarker, interpreted collectively. For example, an altered concentration in at least two, at least three, at least four, at least five, at least six, at least seven, at least eight, at least nine, etc. more biomarkers may be used to diagnose FA. In one embodiment, altered concentrations of more biomarkers increases diagnosis certainty.

The disclosed method integrated metabolic information resulting in one or a panel of biomarkers for FA as a genetic syndrome and FA-associated cancer, to adjust and better target therapies for FA patients. Based on the known DNA repair defects in FA cells, tumors in FA patients are relatively more sensitive to conventional clastogenic treatments, permitting altered treatment such as dose de-escalation of chemotherapeutic agents. The results are applicable to FA patients with SCC and other tumors, and also to a significant proportion of patients with sporadic SCC and other tumors in the general population with mutations in FA or decreased expression of FA, and other DNA repair pathways.

Each of the following are expressly incorporated by reference herein in its entirety:
Alter. Fanconi's anemia and malignancies. Am J Hematol 53 (1996) 99.
Rosenberg et al. Cancer incidence in persons with Fanconi anemia. Blood 101 (2003) 822.
Kutler et al. A 20-year perspective on the International Fanconi Anemia Registry (IFAR). Blood 101 (2003) 1249.
Auerbach. Fanconi anemia and its diagnosis. Mutat Res 668 (2009) 4.
Joenje and Pate. The emerging genetic and molecular basis of Fanconi anaemia. Nat Rev Genet 2 (2001) 446.
Rosenberg et al. How high are carrier frequencies of rare recessive syndromes? Contemporary estimates for Fanconi Anemia in the United States and Israel. Am J Med Genet A 155A (2011) 1877.
Rochowski et al. Patients with Fanconi anemia and AML have different cytogenetic clones than de novo cases of AML. Pediatr Blood Cancer 2012.
Rochowski et al. Estimation of the prevalence of Fanconi anemia among patients with de novo acute myelogenous leukemia who have poor recovery from chemotherapy. Leuk Res 36 (2012) 29.
Garner and Smogorzewska. Ubiquitylation and the Fanconi anemia pathway. FEBS Lett 585 (2011) 2853.
Smogorzewska et al. Identification of the FANCI Protein, a Monoubiquitinated FANCD2 Paralog Required for DNA Repair. Cell 129 (2007) 289.
Taniguchi et al. Convergence of the fanconi anemia and ataxia telangiectasia signaling pathways. Cell 109 (2002) 459.
Sasaki. Is Fanconi's anaemia defective in a process essential to the repair of DNA cross links? Nature 257 (1975) 501.
Auerbach A D. A test for Fanconi's anemia. Blood 1988; 72: 366-367.
Sasaki and Tonomura. A high susceptibility of Fanconi's anemia to chromosome breakage by DNA cross-linking agents. Cancer Res 33 (1973) 1829.
Auerbach. Fanconi anemia diagnosis and the diepoxybutane (DEB) test. Exp Hematol 21 (1993) 731.
Wang. A major switch for the Fanconi anemia DNA damage-response pathway. Nat Struct Mol Bioi 2008; 15: 1128-1130.
Andreassen et al. ATR couples FANCD2 monoubiquitination to the DNA-damage response. Genes Dev 18 (2004) 1958.
Howlett et al. Biallelic inactivation of BRCA2 in Fanconi anemia. Science 297 (2002) 606.
Rahman et al. PALB2, which encodes a BRCA2-interacting protein, is a breast cancer susceptibility gene. Nat Genet 39 (2007) 165.
Xia et al. Fanconi anemia is associated with a defect in the BRCA2 partner PALB2. Nat Genet 39 (2007) 159.
Reid S, Schindler D, Hanenberg H et al. Biallelic mutations in PALB2 cause Fanconi anemia subtype FA-N and predispose to childhood cancer. Nat Genet 2006.
Litman R, Peng M, Jin Z et al. BACH1 is critical for homologous recombination and appears to be the Fanconi anemia gene product FANCJ. Cancer Cell 2005; 8: 255-265.
Levitus M, Waisfisz Q, Godthelp B C et al. The DNA helicase BRIP1 is defective in Fanconi anemia complementation group J. Nat Genet 2005; 37: 934-935.
Levran et al. The BRCA1-interacting helicase BRIP1 is deficient in Fanconi anemia. Nat Genet 37 (2005) 931.
Wang W. Emergence of a DNA-damage response network consisting of Fanconi anaemia and BRCA proteins. Nat Rev Genet 2007; 8: 735-748.
Taniguchi T, Garcia-Higuera I, Andreassen P R et al. S-phase-specific interaction of the Fanconi anemia protein, FANCD2, with BRCA1 and RAD51. Blood 2002; 100:2414-2420.
Garcia-Higuera I, Taniguchi T, Ganesan S et al. Interaction of the Fanconi anemia proteins and BRCA1 in a common pathway. Mol Cell 2001; 7: 249-262.
Roy R, Chun J, Powell S N. BRCA1 and BRCA2: different roles in a common pathway of genome protection. Nat Rev Cancer 2012; 12: 68-78.
Meindl A, Oitsch N, Kast K et al. Hereditary breast and ovarian cancer: new genes, new treatments, new concepts. Dtsch Arztebl Int 2011; 108: 323-330.
Vaz et al. Mutation of the RAD51C gene in a Fanconi anemia-like disorder. Nat Genet 42, 406.
Cantor S B, Guillemette S. Hereditary breast cancer and the BRCA1-associated FANCJ/BACH1/BRIP1. Future Oncol 2011; 7: 253-261.
Tischkowitz M, Xia B. PALB2/FANCN: recombining cancer and Fanconi anemia. Cancer Res 2010; 70: 7353-7359.
Campeau P M, Foulkes W D, Tischkowitz M D. Hereditary breast cancer: new genetic developments, new therapeutic avenues. Hum Genet 2008; 124: 31-42.
Seal et al. Truncating mutations in the Fanconi anemia J gene BRIP1 are low-penetrance breast cancer susceptibility alleles. Nature Genetics 38 (2006) 1239.
van der Heijden M S, Brody J R, Gallmeier E et al. Functional defects in the Fanconi anemia pathway in pancreatic cancer cells. Am J Pathol 2004; 165: 651-657.
Couch F J, Johnson M R, Rabe K et al. Germ line Fanconi anemia complementation group C mutations and pancreatic cancer. Cancer Res 2005; 65: 383-386.
van der Heijden M S, Yeo C J, Hruban R H, Kern S E. Fanconi anemia gene mutations in young-onset pancreatic cancer. Cancer Res 2003; 63: 2585-2588.
Mathew C G. Fanconi anaemia genes and susceptibility to cancer. Oncogene 2006; 25: 5875-5884.
Pejovic T, Yates J E, Liu H Y et al. Cytogenetic instability in ovarian epithelial cells from women at risk of ovarian cancer. Cancer Res 2006; 66: 9017-9025.
Taniguchi et al. Disruption of the Fanconi anemia-BRCA pathway in cisplatin-sensitive ovarian tumors. Nat Med 9 (2003) 568.
Wang Z, Li M, Lu S et al. Promoter hypermethylation of FANCF plays an important role in the occurrence of ovarian cancer through disrupting Fanconi anemia-BRCA pathway. Cancer Bioi Ther 2006; 5: 256-260.
Narayan G, Arias-Pulido H, Nandula S V et al. Promoter hypermethylation of FANCF: disruption of Fanconi Anemia-BRCA pathway in cervical cancer. Cancer Res 2004; 64: 2994-2997.
Marsit C J, Liu M, Nelson H H et al. Inactivation of the Fanconi anemia/BRCA pathway in lung and oral cancers: implications for treatment and survival. Oncogene 2004; 23: 1000-1004.
Ameziane et al. No evidence for FANCF gene silencing in head-and-neck squamous cell carcinomas. Cell Oneal 31 (2009) 53.
Snyder E R, Ricker J L, Chen Z, Waes C V. Variation in cisplatinum sensitivity is not associated with Fanconi Anemia/BRCA pathway inactivation in head and neck squamous cell carcinoma cell lines. Cancer Lett 2007; 245: 75-80.
Burkitt K, Ljungman M. Compromised Fanconi anemia response due to BRCA1 deficiency in cisplatin-sensitive head and neck cancer cell lines. Cancer Lett 2007; 253: 131-137.
Meetei A R, de Winter J P, Medhurst A L et al. A novel ubiquitin ligase is deficient in Fanconi anemia. Nat Genet 2003; 35: 165-170.
Meetei A R, Levitus M, Xue Y et al. X-linked inheritance of Fanconi anemia complementation group B. Nat Genet 2004; 36: 1219-1224.
Meetei A R, Medhurst A L, Ling C et al. A human ortholog of archaeal DNA repair protein Hef is defective in Fanconi anemia complementation group M. Nat Genet 2005; 37: 958-963.
de Winter et al. Isolation of a eDNA representing the Fanconi anemia complementation group E gene. Am J Hum Genet 67 (2000) 1306.
de Winter J P, Waisfisz Q, Rooimans M A et al. The Fanconi anaemia group G gene FANCG is identical with XRCC9. Nat Genet 1998; 20: 281-283.
Lo Ten Foe J R, Rooimans M A, Bosnoyan-Collins L et al. Expression cloning of a eDNA for the major Fanconi anaemia gene, FAA. Nat Genet 1996; 14: 320-323.
Strathdee C A, Gavish H, Shannon W R, Buchwald M. Cloning of cDNAs for Fanconi's anaemia by functional complementation. Nature 1992; 358: 434.
Timmers et al. Positional cloning of a novel Fanconi anemia gene, FANCD2. Mol Cell 7 (2001) 241.
de Winter J P, Rooimans M A, van Der Weel L et al. The Fanconi anaemia gene FANCF encodes a novel protein with homology to ROM. Nat Genet 2000; 24: 15-16.
Sims A E, Spiteri E, Sims R J, 3rd et al. FANCI is a second monoubiquitinated member of the Fanconi anemia pathway. Nat Struct Mol Bioi 2007.

Positional cloning of the Fanconi anaemia group A gene. Nat Genet 1996; 14: 324-328.
Dorsman J C, Levitus M, Rockx D et al. Identification of the Fanconi anemia complementation group I gene, FANCI. Cell Oneal 2007; 29: 211-218.
Vaz F, Hanenberg H, Schuster B et al. Mutation of the RAD51C gene in a Fanconi anemia-like disorder. Nat Genet 2010; 42: 406-409.
Stoepker et al. SLX4, a coordinator of structure-specific endonucleases, is mutated in a new Fanconi anemia subtype. Nat Genet 43 (2011) 138.
Kim Y, Lach F P, Desetty R et al. Mutations of the SLX4 gene in Fanconi anemia. Nat Genet 2011; 43: 142-146.
Hoskins E E, Gunawardena R W, Habash K B et al. Coordinate regulation of Fanconi anemia gene expression occurs through the Rb/E2F pathway. Oncogene 2008; 27: 4798-4808.
Ciccia A, Ling C, Coulthard R et al. Identification of FAAP24, a Fanconi anemia core complex protein that interacts with FANCM. Mol Cell 2007; 25: 331-343.
Ling et al. FAAP100 is essential for activation of the Fanconi anemia-associated DNA damage response pathway. Embo J 26 (2007) 2104.
Moldovan G L, D'Andrea A D. How the Fanconi anemia pathway guards the genome. Annu Rev Genet 2009; 43: 223-249.
Ali A M, Pradhan A, Singh T R et al. FAAP20: a novel ubiquitin-binding FA nuclear core-complex protein required for functional integrity of the FA-BRCA DNA repair pathway. Blood 2012; 119: 3285-3294.
Leung J W, Wang Y, Fang K W et al. Fanconi anemia (FA) binding protein FAAP20 stabilizes FA complementation group A (FANCA) and participates in interstrand cross-link repair. Proc Natl Acad Sci USA 2012; 109: 4491-4496.
Collins N B, Wilson J B, Bush T et al. ATR-dependent phosphorylation of FANCA on serine 1449 after DNA damage is important for FA pathway function. Blood 2009; 113: 2181-2190.
Qiao F, Mi J, Wilson J B et al. Phosphorylation of fanconi anemia (FA) complementation group G protein, FANCG, at serine 7 is important for function of the FA pathway. J Bioi Chem 2004; 279: 46035-46045.
Ho et al. Phosphorylation of FANCD2 on two novel sites is required for mitomycin C resistance. Mol Cell Bioi 26 (2006) 7005.
Matsuoka S, Ballif B A, Smogorzewska A et al. ATM and ATR substrate analysis reveals extensive protein networks responsive to DNA damage. Science 2007; 316: 1160-1166.
Machida Y J, Machida Y, Chen Y et al. UBE2T is the E2 in the Fanconi anemia pathway and undergoes negative autoregulation. Mol Cell 2006; 23: 589-596.
Cole A R, Lewis L P C, Walden H. The structure of the catalytic subunit FANCL of the Fanconi anemia core complex. Nature Structural & Molecular Biology 2010; 17: 294-U254.
Singh et al. Impaired FANCD2 monoubiquitination and hypersensitivity to camptothecin uniquely characterize Fanconi anemia complementation group M. Blood 114 (2009) 174.
Singh T R, Sara D, Ali A M et al. MHF1-MHF2, a histone-fold-containing protein complex, participates in the Fanconi anemia pathway via FANCM. Mol Cell 2010; 37: 879-886.
Kim J M, Kee Y, Gurtan A, D'Andrea A D. Cell cycle-dependent chromatin loading of the Fanconi anemia core complex by FANCM/FAAP24. Blood 2008; 111: 5215-5222.

Gari K D C, Delannoy M, Wu L, Constantinou A. Remodeling of DNA replication structures by the branch point translocase FANCM. Proc. Natl. Acad. Sci. USA 2008; 105: 16107-16112.

Hovest M G, Krieg T, Herrmann G. Differential roles for Chk1 and FANCD2 in ATR-mediated signalling for psoralen photoactivation-induced senescence. Exp Dermatol 2011; 20: 883-889.
Ishiai M, Kitao H, Smogorzewska A et al. FANCI phosphorylation functions as a molecular switch to turn on the Fanconi anemia pathway. Nat Struct Mol Bioi 2008; 15: 1138-1146.
Takata et al. The Fanconi anemia pathway: insights from somatic cell genetics using DT40 cell line. Mutat Res 668 (2009) 92.
Cole A R, Lewis L P, Walden H. The structure of the catalytic subunit FANCL of the Fanconi anemia core complex. Nat Struct Mol Bioi 2010; 17: 294-298.
Hoskins E E, Morris T A, Higginbotham J M et al. Fanconi anemia deficiency stimulates HPV-associated hyperplastic growth in organotypic epithelial raft culture. Oncogene 2009; 28: 674-685.
MacKay C, Declais A C, Lundin C et al. Identification of KIAA1018/FAN1, a DNA repair nuclease recruited to DNA damage by monoubiquitinated FANCD2. Cell 2010; 142: 65-76.
Smogorzewska et al. A genetic screen identifies FAN1, a Fanconi anemia-associated nuclease necessary for DNA interstrand crosslink repair. Mol Cell 39 (2010) 36.
O'Donnell L, Durocher D. DNA repair has a new FAN1 club. Mol Cell 2010; 39: 167-169.
Liu T, Ghosal G, Yuan J et al. FAN1 acts with FANCI-FANCD2 to promote DNA interstrand cross-link repair. Science 2010; 329: 693-696.
Wang X, Andreassen P R, D'Andrea AD. Functional interaction of monoubiquitinated FANCD2 and BRCA2/FANCD1 in chromatin. Mol Cell Biol 2004; 24: 5850-5862.
Howlett N G, Taniguchi T, Durkin S G et al. The Fanconi anemia pathway is required for the DNA replication stress response and for the regulation of common fragile site stability. Hum Mol Genet 2005; 14:693-701.
Deans A J, West S C. DNA interstrand crosslink repair and cancer. Nat Rev Cancer 2011; 11: 467-480.
Nijman et al. The deubiquitinating enzyme USP1 regulates the Fanconi anemia pathway. Mol Cell 17 (2005) 331.
Oestergaard V H, Langevin F, Kuiken H J et al. Deubiquitination of FANCD2 is required for DNA crosslink repair. Mol Cell 2007; 28: 798-809.
Kim J M, Parmar K, Huang M et al. Inactivation of murine Usp1 results in genomic instability and a Fanconi anemia phenotype. Dev Cell 2009; 16: 314-320.
Alter B P. Cancer in Fanconi anemia, 1927-2001. Cancer 2003; 97: 425-440.
D'Andrea and Grompe. The Fanconi anaemia/BRCA pathway. Nat Rev Cancer 3 (2003) 23.
Kennedy R D, D'Andrea A D. The Fanconi Anemia/BRCA pathway: new faces in the crowd. Genes Dev 2005; 19: 2925-2940.
MacMillan M L, Hughes M R, Agarwal S, Daley G Q. Cellular therapy for fanconi anemia: the past, present, and future. Bioi Blood Marrow Transplant 17: S109-114.
Schultz J C, Shahidi N T. Tumor necrosis factor-alpha overproduction in Fanconi's anemia. Am J Hematol 1993; 42: 196-201.
Rosselli F, Sanceau J, Gluckman E et al. Abnormallymphokine production: a novel feature of the genetic disease Fanconi anemia. II. In vitro and in vivo spontaneous overproduction of tumor necrosis factor alpha. Blood 1994; 83: 1216-1225.
Dufour C, Carcione A, Svahn J et al. TNF-alpha and IFN-gamma are overexpressed in the bone marrow of Fanconi anemia patients and TNF-alpha suppresses erythropoiesis in vitro. Blood 2003; 102: 2053-2059.
Lensch et al. Selective pressure as an essential force in molecular evolution of myeloid leukemic clones: a view from the window of Fanconi anemia. Leukemia 13 (1999) 1784.
Myers K C, Davies S M. Hematopoietic stem cell transplantation for bone marrow failure syndromes in children. Bioi Blood Marrow Transplant 2009; 15: 279-292.
Alter B P. Bone marrow failure syndromes in children. Pediatr Clin North Am 2002; 49: 973-988.
Tischkowitz M, Dokal I. Fanconi anaemia and leukaemia—clinical and molecular aspects. Br J Haematol 2004; 126: 176-191.
Butturini et al. Hematologic abnormalities in Fanconi anemia: an International Fanconi Anemia Registry study. Blood 84 (1994) 1650.
Wagner et al. Germline mutations in BRCA2: shared genetic susceptibility to breast cancer, early onset leukemia, and Fanconi anemia. Blood 103 (2004) 3226.
Hirsch et al. Association of biallelic BRCA2/FANCD1 mutations with spontaneous chromosomal instability and solid tumors of childhood. Blood 103 (2004) 2554.
Tischkowitz et al. Bi-allelic silencing of the Fanconi anaemia gene FANCF in acute myeloid leukaemia. Br J Haematol 123 (2003) 469.
Hess et al. Hypermethylation of the FANCC and FANCL promoter regions in sporadic acute leukaemia. Cell Oneal 30 (2008) 299.
Rosenberg et al. Cancer risks in Fanconi anemia: findings from the German Fanconi Anemia Registry. Haematologica 93 (2008) 511.
Mehta et al. Numerical chromosomal changes and risk of development of myelodysplastic syndrome—acute myeloid leukemia in patients with Fanconi anemia. Cancer Genet Cytogenet 203 (2010) 180.
D'Andrea. Targeting DNA repair pathways in AML. Best Pract Res Clin Haematol 23 (2010) 469.
Forastiere et al. Head and neck cancer. N Engl J Med 345 (2001) 1890.
Leemans C R, Braakhuis B J, Brakenhoff R H. The molecular biology of head and neck cancer. Nat Rev Cancer 2011; 11: 9-22.
Califano et al. Genetic progression model for head and neck cancer: implications for field cancerization. Cancer Res 56 (1996) 2488.
Jemal A, Siegel R, Xu J, Ward E. Cancer statistics, 2010. CA Cancer J Clin 2010; 60: 277-300.
Baron A E, Franceschi S, Barra S et al. A comparison of the joint effects of alcohol and smoking on the risk of cancer across sites in the upper aerodigestive tract. Cancer Epidemiol Biomarkers Prev 1993; 2: 519-523.
Parkin D M. Global cancer statistics in the year 2000. Lancet Oneal 2001; 2: 533-543.
Field J K. Oncogenes and tumour-suppressor genes in squamous cell carcinoma of the head and neck. Eur J Cancer B Oral Oncol 1992; 28B: 67-76.
de Araujo M R, Rubira-Bullen I R, Santos C F et al. High prevalence of oral human papillomavirus infection in Fanconi's anemia patients. Oral Dis 2011; 17: 572-576.
Kutler et al. Human papillomavirus DNA and p53 polymorphisms in squamous cell carcinomas from Fanconi anemia patients. J Natl Cancer Inst 95 (2003) 1718.
van Zeeburg H J, Snijders P J, Wu T et al. Clinical and molecular characteristics of squamous cell carcinomas from Fanconi anemia patients. J Natl Cancer Inst 2008; 100: 1649-1653.
Li F P, Montesano R. Interactions of cancer susceptibility genes and environmental carcinogens. American Association for Cancer Research (AACR)-International Agency for Research on Cancer (IARC) Joint Conference. Cancer Res 1994; 54: 4243-4247.
Kutler D I, Auerbach A D, Satagopan J et al. High incidence of head and neck squamous cell carcinoma in patients with Fanconi anemia. Arch Otolaryngol Head Neck Surg 2003; 129: 106-112.
Cloos et al. Genetic susceptibility to head and neck squamous cell carcinoma. J Natl Cancer Inst 88 (1996) 530.
Birkeland A C, Auerbach A D, Sanborn E et al. Postoperative clinical radiosensitivity in patients with fanconi anemia and head and neck squamous cell carcinoma. Arch Otolaryngol Head Neck Surg 2011; 137: 930-934.
Alter B P. Radiosensitivity in Fanconi's anemia patients. Radiother Oneal 2002; 62: 345-347.
Bremer M, Schindler D, Gross Metal. Fanconi's anemia and clinical radiosensitivity report on two adult patients with locally advanced solid tumors treated by radiotherapy. Strahlenther Onkol 2003; 179: 748-753.
Spanier G, Pohl F, Giese T et al. Fatal course of tonsillar squamous cell carcinoma associated with Fanconi anaemia: A mini review. J Craniomaxillofac Surg 2011.
Sparano A, Quesnelle K M, Kumar M S et al. Genome-wide profiling of oral squamous cell carcinoma by array-based comparative genomic hybridization. Laryngoscope 2006; 116: 735-741.
Stransky et al. The mutational landscape of head and neck squamous cell carcinoma. Science 333 (2011) 1157.
Agrawal N, Frederick M J, Pickering C R et al. Exome sequencing of head and neck squamous cell carcinoma reveals inactivating mutations in NOTCH1. Science 2011; 333: 1154-1157.
Rosenberg P S, Socie G, Alter B P, Gluckman E. Risk of head and neck squamous cell cancer and death in patients with Fanconi anemia who did and did not receive transplants. Blood 2005; 105: 67-73.
Kaplan M J, Sabia H, Wanebo H J, Cantrell R W. Squamous cell carcinoma in the immunosuppressed patient: Fanconi's anemia. Laryngoscope 1985; 95: 771-775.
Lyakhovich and Surralles. Disruption of the Fanconi anemia/BRCA pathway in sporadic cancer. Cancer Lett 232 (2006) 99.
Valeri A, Martinez S, Casado J A, Bueren J A. Fanconi anaemia: from a monogenic disease to sporadic cancer. Clin Transl Oneal 2011; 13: 215-221.
Wei M, Xu J, Dignam J et al. Estrogen receptor alpha, BRCA1, and FANCF promoter methylation occur in distinct subsets of sporadic breast cancers. Breast Cancer Res Treat 2007.
Russell P A, Pharoah P D, De Fay K et al. Frequent loss of BRCA1 mRNA and protein expression in sporadic ovarian cancers. Int J Cancer 2000; 87: 317-321.
Thompson M E, Jensen R A, Obermiller P S et al. Decreased expression of BRCA1 accelerates growth and is often present during sporadic breast cancer progression. Nat Genet 1995; 9: 444-450.
Wreesmann V B, Estilo C, Eisele D W et al. Downregulation of Fanconi anemia genes in sporadic head and neck squamous cell carcinoma. ORL J Otorhinolaryngol Relat Spec 2007; 69: 218-225.
Tremblay S, Pintar Dos Reis P, Bradley G et al. Young patients with oral squamous cell carcinoma: study of the involvement of GSTP1 and deregulation of the Fanconi anemia genes. Arch Otolaryngol Head Neck Surg 2006; 132: 958-966.
Dhillon V S, Shahid M, Husain S A. CpG methylation of the FHIT, FANCF, cyclin-D2, BRCA2 and RUNX3 genes in Granulosa cell tumors (GCTs) of ovarian origin. Mol Cancer 2004; 3: 33.
Szaumkessel M, Richter J, Giefing M et al. Pyrosequencing-based DNA methylation profiling of Fanconi anemia/BRCA pathway genes in laryngeal squamous cell carcinoma. Int J Oneal 2011; 39: 505-514.
Wang S S, Bratti M C, Rodriguez A C et al. Common variants in immune and DNA repair genes and risk for human papillomavirus persistence and progression to cervical cancer. J Infect Dis 2009; 199: 20-30.
Braakhuis et al. Molecular analysis of surgical margins in head and neck cancer: more than a marginal issue. Oral Oncol 46 (2010) 485.
Braakhuis B J, Tabor M P, Kummer J A et al. A genetic explanation of Slaughter's concept of field cancerization: evidence and clinical implications. Cancer Res 2003; 63: 1727-1730.
Li J, Sejas D P, Zhang X L et al. TNF-alpha induces leukemic clonal evolution ex vivo in Fanconi anemia group C murine stem cells. Journal of Clinical Investigation 2007; 117: 3283-3295.
Meyer S, Neitzel H, Tonnies H. Chromosomal aberrations associated with clonal evolution and leukemic transformation in fanconi anemia: clinical and biological implications. Anemia 2012; 2012: 349837.

Gillison M L, Koch W M, Capone R B et al. Evidence for a causal association between human papillomavirus and a subset of head and neck cancers. J Natl Cancer Inst 2000; 92: 709-720.
Kreimer A R, Clifford G M, Boyle P, Franceschi S. Human papillomavirus types in head and neck squamous cell carcinomas worldwide: a systematic review. Cancer Epidemiol Biomarkers Prey 2005; 14: 467-475.
Schlecht N F, Burk R D, Adrien L et al. Gene expression profiles in HPV-infected head and neck cancer. J Pathol 2007; 213: 283-293.
Adelstein O J, Ridge J A, Gillison M L et al. Head and neck squamous cell cancer and the human papillomavirus: summary of a National Cancer Institute State of the Science Meeting, Nov. 9-10, 2008, Washington, D.C. Head Neck 2009; 31: 1393-1422.
Chaturvedi A K, Engels E A, Pfeiffer R M et al. Human papillomavirus and rising oropharyngeal cancer incidence in the United States. J Clin Oncol 2011; 29: 4294-4301.
Gillison M L, Shah K V. Chapter 9: Role of mucosal human papillomavirus in nongenital cancers. J Natl Cancer Inst Monogr 2003; 57-65.
Herrero Chapter 7: Human papillomavirus and cancer of the upper aerodigestive tract. J Natl Cancer Inst Monogr 2003, 47.
zur Hausen H. Papillomaviruses and cancer: from basic studies to clinical application. Nat Rev Cancer 2002; 2: 342-350.
Munger K, Phelps W C, Bubb V et al. The E6 and E7 genes of the human papillomavirus type 16 together are necessary and sufficient for transformation of primary human keratinocytes. J Virol 1989; 63: 4417-4421.
Hawley-Nelson P, Vousden K H, Hubbert N L et al. HPV16 E6 and E7 proteins cooperate to immortalize human foreskin keratinocytes. EMBO J 1989; 8: 3905-3910.
Pecoraro G, Morgan D, Defendi V. Differential effects of human papillomavirus type 6, 16 and 18 DNAs on immortalization and transformation of human cervical epithelial cells. Proc Natl Acad Sci USA 1989.
Woodworth C D, Doninger J, DiPaolo J A. Immortalization of human foreskin keratinocytes by various human papillomavirus DNAs corresponds to their association with cervical carcinoma. J. Virol. 1989; 63: 159-164.
McCance O J, Kopan R, Fuchs E, Laimins L A. Human papillomavirus type 16 alters human epithelial cell differentiation in vitro. Proc Natl Acad Sci USA 1988; 85: 7169-7173.
Syrjanen S. Human papillomavirus (HPV) in head and neck cancer. J Clin Virol 2005; 32 Suppl 1: S59-66.
Snijders P J, Cromme F V, van den Brule A J et al. Prevalence and expression of human papillomavirus in tonsillar carcinomas, indicating a possible viral etiology. Int J Cancer 1992; 51: 845-850.
Spardy N, Duensing A, Charles D et al. The human papillomavirus type 16 E7 oncoprotein activates the Fanconi anemia (FA) pathway and causes accelerated chromosomal instability in FA cells. J Virol 2007; 81: 13265-13270.
Munger et al. Mechanisms of human papillomavirus-induced oncogenesis. J Virol 78 (2004) 11451.
Longworth M S, Laimins L A. Pathogenesis of human papillomaviruses in differentiating epithelia. Microbial Mol Bioi Rev 2004; 68: 362-372.
DiMaio D, Liao J B. Human papillomaviruses and cervical cancer. Adv Virus Res 2006; 66: 125-159.
Klingelhutz A J, Qian Q, Phillips S L et al. Amplification of the chromosome 20q region is associated with expression of HPV-16 E7 in human airway and anogenital epithelial cells. Virology 2005; 340: 237-244.
Wang J, Sampath A, Raychaudhuri P, Bagchi S. Both Rb and E7 are regulated by the ubiquitin proteasome pathway in HPV-containing cervical tumor cells. Oncogene 2001; 20: 4740-4749.
Reinstein E, Scheffner M, Oren M et al. Degradation of the E7 human papillomavirus oncoprotein by the ubiquitin-proteasome system: targeting via ubiquitination of the N-terminal residue. Oncogene 2000; 19: 5944-5950.
Lin CHI Chang H S, Yu we. USP11 stabilizes HPV-16E7 and further modulates the E7 biological activity. J Bioi Chem 2008; 283: 15681-15688.
van Zeeburg H J, Snijders P J, Pals G et al. Generation and molecular characterization of head and neck squamous cell lines of fanconi anemia patients. Cancer Res 2005; 65: 1271-1276.
Hickman E S, Moroni M C, Helin K. The role of p53 and pRB in apoptosis and cancer. Curr Opin Genet Dev 2002; 12: 60-66.
Hoskins E E, Morreale R J, Werner S P et al. The Fanconi Anemia Pathway Limits Human Papillomavirus Replication. J Virol 2012.
Wadsworth J T, Somers K D, Cazares L H et al. Serum protein profiles to identify head and neck cancer. Clin Cancer Res 2004; 10: 1625-1632.
Nagaraj N S. Evolving ‘omics’ technologies for diagnostics of head and neck cancer. Brief Funct Genomic Proteomic 2009; 8: 49-59.
Zimmermann B G, Park N J, Wong D T. Genomic targets in saliva. Ann N Y Acad Sci 2007; 1098: 184-191.
Willner D, Furlan M, Schmieder R et al. Metagenomic detection of phage-encoded platelet-binding factors in the human oral cavity. Proc Natl Acad Sci USA 2011; 108 Suppl 1: 4547-4553.

Crielaard W, Zaura E, Schuller A A et al. Exploring the oral microbiota of children at various developmental stages of their dentition in the relation to their oral health. BMC Med Genomics 2011; 4: 22.
Burbelo P D, Bayat A, Lebovitz E E, Iadarola M J. New technologies for studying the complexity of oral diseases. Oral Dis 2012; 18: 121-126.
Hu S, Arellano M, Boontheung P et al. Salivary proteomics for oral cancer biomarker discovery. Clin Cancer Res 2008; 14: 6246-6252.
Remmerbach T W, Maurer K, Janke S et al. Oral brush biopsy analysis by matrix assisted laser desorption/ionisation-time of flight mass spectrometry profiling—a pilot study. Oral Oncol 2011; 47: 278-281.
Nicholson et al. ‘Metabonomics’: understanding the metabolic responses of living systems to pathophysiological stimuli via multivariate statistical analysis of biological NMR spectroscopic data. Xenobiotica 29 (1999) 1181.
Fan et al. The promise of metabolomics in cancer molecular therapeutics. Curr Opin Mol Ther 6 (2004) 584.
Giskeodegard et al. Multivariate modeling and prediction of breast cancer prognostic factors using MR metabolomics. J Proteome Res 9 (2010) 972.
Yang et al. Metabonomic studies of human hepatocellular carcinoma using high-resolution magic-angle spinning 1H NMR spectroscopy in conjunction with multivariate data analysis. J Proteome Res 6 (2007) 2605.
Nicholson J K, Connelly J, Lindon J C, Holmes E. Metabonomics: a platform for studying drug toxicity and gene function. Nat Rev Drug Discov 2002; 1: 153-161.
Pan Z, Raftery D. Comparing and combining NMR spectroscopy and mass spectrometry in metabolomics. Anal Bioanal Chem 2007; 387: 525-527.
Zhang et al. Esophageal cancer metabolite biomarkers detected by LC-MS and NMR methods. PLoS One 7 (2012) e30181.
Napoli et al. Urine metabolic signature of pancreatic ductal adenocarcinoma by (1)h nuclear magnetic resonance: identification, mapping, and evolution. J Proteome Res 11 (2012) 1274.
Bu et al. Metabolomics: A Revolution for Novel Cancer Marker Identification. Combinatorial Chemistry & High Throughput Screening, Volume 15, Number 3, March 2012, pp. 266-275(10).
Ma et al. Metabolomics in the fields of oncology: a review of recent research. July 2012, Volume 39, Issue 7, pp 7505-7511.
Kim H, D'Andrea AD. Regulation of DNA cross-link repair by the Fanconi anemia/BRCA pathway. Genes Dev. 2012; 26(13):1393-408.3403008.
Kottemann and Smogorzewska. Fanconi anaemia and the repair of Watson and Crick DNA crosslinks. Nature. 493 (2013) 356.
Alter Fanconi's anemia and malignancies. Am J. Hematol. 53 (1996) 99.
Rosenberg et al. Cancer incidence in persons with Fanconi anemia. Blood. 101 (2003) 822.
Kutler et al. A 20-year perspective on the International Fanconi Anemia Registry (IFAR). Blood. 101 (2003) 1249.
Auerbach. Fanconi anemia and its diagnosis. Mutat Res. 2009; 668(1-2):4-10.2742943.
Montes de Oca et al. Regulated interaction of the Fanconi anemia protein, FANCD2, with chromatin. Blood. 105 (2005) 1003.
Zhang et al. FANCJ/BRIP1 recruitment and regulation of FANCD2 in DNA damage responses. Chromosome. 119 (2010) 637.
Song et al. A novel role for Fanconi anemia (FA) pathway effector protein FANCD2 in cell cycle progression of untransformed primary human cells. Cell Cycle. 9 (2010) 2375.
Bakker et al. Learning from a paradox: recent insights into Fanconi anaemia through studying mouse models. Dis Model Mech. 6 (2013) 40.
Rochowski et al. Patients with Fanconi anemia and AML have different cytogenetic clones than de novo cases of AML. Pediatr Blood Cancer. 59 (2012) 922.
Rochowski et al. Estimation of the prevalence of Fanconi anemia among patients with de novo acute myelogenous leukemia who have poor recovery from chemotherapy. Leuk Res. 36 (2012) 29.
Rosenberg et al. Cancer risks in Fanconi anemia: findings from the German Fanconi Anemia Registry. Haematologica. 93 (2008) 511.
Alter. Cancer in Fanconi anemia, 1927-2001. Cancer. 97 (2003) 425.
Birkeland et al. Postoperative clinical radiosensitivity in patients with fanconi anemia and head and neck squamous cell carcinoma. Arch Otolaryngol Head Neck Surg. 137 (2011) 930.
Nicholson et al. ‘Metabonomics’: understanding the metabolic responses of living systems to pathophysiological stimuli via multivariate statistical analysis of biological NMR spectroscopic data. Xenobiotica. 29 (1999) 1181.
Nicholson et al. Metabonomics: a platform for studying drug toxicity and gene function. Nat Rev Drug Discov. 1 (2002) 153.
Fan et al. The promise of metabolomics in cancer molecular therapeutics. Curr Opin Mol Ther. 6 (2004) 584.
Giskeodegard et al. Multivariate modeling and prediction of breast cancer prognostic factors using MR metabolomics. J Proteome Res. 9 (2010) 972.
Yang et al. Metabonomic studies of human hepatocellular carcinoma using high-resolution magic-angle spinning 1H NMR spectroscopy in conjunction with multivariate data analysis. J Proteome Res. 6 (2007) 2605.
Goodpaster et al. Statistical significance analysis of nuclear magnetic resonance-based metabonomics data. Anal Biochem. 401 (2010) 134.
Hoskins et al. Fanconi anemia deficiency stimulates HPV-associated hyperplastic growth in organotypic epithelial raft culture. Oncogene. 28 (2009) 674.
Hoskins et al. The Fanconi Anemia Pathway Limits Human Papillomavirus Replication. J. Virol. 86 (2012) 8131.
Romick-Rosendale et al. NMR-based metabonomics analysis of mouse urine and fecal extracts following oral treatment with the broad-spectrum antibiotic enrofloxacin (Baytril). Magn Reson Chem. 47 (2009) S36.
Romick-Rosendale et al. The Fanconi anemia pathway: Repairing the link between DNA damage and squamous cell carcinoma. Mutat Res. 743 (2013) 78.

The embodiments shown and described in the specification are only specific embodiments of inventors who are skilled in the art and are not limiting in any way. Therefore, various changes, modifications, or alterations to those embodiments may be made without departing from the spirit of the invention in the scope of the following claims. The references cited are expressly incorporated by reference herein in their entirety.

What is claimed is:

1. A method for adjusting therapy given to a patient at risk of or having cancer, the method comprising diagnosing Fanconi Anemia (FA) and/or predicting bone marrow failure (BMF) in a patient by determining a level of at least one biomarker selected from the group consisting of 3-hydroxyisovalerate, acetate, isopropanol, glycerol-3-phosphocholine, choline, histamine, glutamate, alanine, glycine, isoleucine, lysine, phenylalanine, threonine, tyrosine, vitamin A, vitamin D, cholesterol, ketone bodies, acetone, acetoacetate, and acetoacetaldehyde where the level of the biomarker is assessed in at least one biological sample obtained from the patient and where an altered level of the biomarker compared to a healthy control patient is indicative of FA and/or predictive of BMF, and adjusting the patient therapy based on the diagnosis of FA and/or prediction of BMF in the patient.

2. The method of claim 1 where the patient is metabolically stressed prior to determining the biomarker level.

3. The method of claim 1 where the biomarker has a role in or is affected by lipid metabolism, and the patient is fasted and/or the patient has induced ketogenesis prior to obtaining the biological sample.

4. The method of claim 1 where the biomarker is at least one of 3-hydroxyisovalerate, acetate, isopropanol, glycerol-3-phosphocholine, phosphocholine, choline, and histamine, and the patient having cancer is diagnosed with FA when the level of the biomarker is greater than in the healthy control patient.

5. The method of claim 1 where the biomarker is at least one of glutamate, alanine, glycine, isoleucine, lysine, phenylalanine, threonine, and tyrosine, and the patient having cancer is diagnosed with FA when the level of the biomarker is lower than in the healthy control patient.

6. The method of claim 1 where the cancer is a solid tumor.

7. The method of claim 6 where the cancer is squamous cell carcinoma (SSC).

8. The method of claim 1 where the patient also has bone marrow failure (BMF) or leukemia.

9. The method of claim 1 where a confidence level of the diagnosis of FA is increased when the number of biomarkers having altered levels is increased.

10. The method of claim 1 where the at least one biomarker level is determined by comparing nuclear magnetic resonance (NMR) peak size of at least one biomarker analyte in a biological sample from a FA patient compared to a corresponding NMR peak size of the biomarker analyte in the same type of biological sample from a control.

11. The method of claim 10 where the biological sample is selected from the group consisting of plasma, serum, urine, feces, cerebrospinal fluid, saliva, lavage fluid, intrathecal fluid, and combinations thereof.

12. The method of claim 10 where the biological sample is urine.

13. The method of claim 12 where at least acetone and acetoacetate/acetaldehyde are biomarkers.

14. The method of claim 10 wherein the biological sample is saliva and the control is a genetically related family member without FA of the FA patient, or a healthy subject.

15. The method of claim 1 where the at least one biomarker level is determined by a clinical assay.

16. The method of claim 1 where a plurality of biomarkers comprising a biomarker signature is determined.

17. The method of claim 16 where the biomarker signature comprises components of or affected by lipid metabolism.

18. The method of claim 17 where the biomarker signature comprises components of or affected by cholesterol metabolism.

19. A method for identifying an individual as a carrier of Fanconi Anemia (FA), the method comprising
assessing an increased level of a hippurate and/or phenylalanine biomarker in a biological sample of the individual compared to the level in the biological sample of a healthy control patient, indicating a FA carrier status of the individual, and
providing at least one of genetic counseling during pregnancy, newborn adjusted care, pre- and/or post-implantation screens of embryos to the individuals based on the identification.

20. The method of claim 19 where the individual identified as a carrier of FA is heterozygous for an FA-dependent gene and/or does not exhibit symptoms of FA.
